SEQUENCE LISTING (4 sequences)

SEQ ID NO: 1 - protein, 26 aa, Artificial Sequence
  Description of Artificial Sequence Synthetic peptide motif
XXCXXXXCXXXXXXXXXLXXHXXXXH

SEQ ID NO: 2 - protein, 56 aa, Artificial Sequence
  Description of Artificial Sequence Synthetic peptide motif
CXXCXXXXXXXXXXXXXCXXCXXXXXXXXXXXXXXXCXXXXXCXXXXXXXXXCXXC

SEQ ID NO: 3 - protein, 32 aa, Artificial Sequence
  Description of Artificial Sequence Synthetic peptide motif
KRXXXRKSAQNXXSAQSAQXRKTAENQXRXRK

SEQ ID NO: 4 - protein, 25 aa, Artificial Sequence
  Description of Artificial Sequence Synthetic peptide motif
CXXCXXXXXXXXXXXXXXXXXCXXC

(19) United States
(12) Patent Application Publication
(10) Pub. No.: US 2005/0079512 A1
(43) Pub. Date: Apr. 14, 2005

(54) Methods of modulating gene expression

(76) Inventors: Beverly M. Emerson, San Diego, CA (US); Shilpa B. Kadam, San Diego, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/783,672

(22) Filed: Feb. 20, 2004

Related U.S. Application Data
(60) Provisional application No. 60/450,771, filed on Feb. 26, 2003.

(51) Int. Cl. C12Q 1/68; C12N 15/85

(52) U.S. Cl. 435/6; 435/455

(57) ABSTRACT

Provided herein are methods and compounds for altering remodeling of chromatin in a cell, and thereby modulating gene expression in the cell.

### RELATED PATENT APPLICATION

This patent application claims the benefit of U.S. patent application No. 60/450,771 filed Feb. 26, 2003, entitled “Methods of modulating gene expression,” naming Beverly M. Emerson and Shilpa B. Kadam as inventors, and bearing attorney docket no. 532793000300. This patent application is incorporated herein by reference in its entirety.

### STATEMENT OF GOVERNMENT SUPPORT

This invention was made in part with government support under Grant No. GM-38760 awarded by the National Institutes of Health. The government has certain rights in this invention.

### FIELD OF THE INVENTION

The methods and compositions provided herein relate generally to the regulation of gene expression. In particular, the methods identify compounds for the modulation of gene expression through targeted chromatin remodeling.

### BACKGROUND

Appropriate differentiation and development of higher organisms require precisely regulated expression of multiple genes. The primary control for most genes is exerted at the level of transcription. This involves the combinatorial action of tissue-specific and ubiquitous transcription factors acting at regulatory sequences that are proximal (promoters) or distal (enhancers, insulators, silencers, and locus control regions [LCRs]) to a gene. The existence of functionally distinct cis-acting elements indicates that the high degree of regulation involved in coordinated gene expression within a complex organism requires more intricate circuitry than a simple promoter can provide to turn genes on and off. A critical aspect of this circuitry and coordination is the regulation imposed upon genes within a complex nuclear environment.

The human genome is composed of about 3.3×10⁹ bp. If stretched out, this would represent a length of more than 1.8 meters of DNA. The cell nucleus that contains two copies of this DNA is, on the other hand, a sphere of no more than 6 μm in diameter. To reach this high level of compaction, human DNA is, like in all other eukaryotes, organized into chromatin. The packaging of DNA into chromatin within the eukaryotic nucleus is highly organized and plays a critical role in regulating gene expression and other nuclear processes. The basic structural unit of chromatin is the nucleosome, which consists of ˜146 bp of DNA wrapped in 1.75 superhelical turns around a histone octamer containing two molecules each of histones H2A, H2B, H3 and H4. This unit is repeated once every 200+/−40 bp as a nucleosomal array in chromosomal DNA. The array is further compacted into a higher-order structure by the association of histone H1 with nucleosomes within the array.

The functional consequence of chromatin packaging, in general, is to restrict access of the DNA to a variety of DNA-binding proteins that regulate gene activity. Biochemical and genetic evidence amply demonstrate that nucleosomes are normally repressive for transcription. Several mechanisms have evolved that modulate chromatin structure to increase the accessibility of DNA for protein interaction. These pathways involve distinct protein complexes that function either as motors to disrupt nucleosomes (ATP-driven chromatin remodeling complexes) or as enzymatic machinery to chemically modify histones (histone acetyltransferases and deacetylases). Such mechanisms may be critical in programming genes to be either active or inactive in a particular cell type or to be poised for expression at a specific stage of development or in response to environmental signals.

Chromatin structural changes can occur at several levels: either globally by the decondensation (active nucleic acids) or condensation (inactive nucleic acids) of a large chromosomal domain or locally by the disruption (active) or formation (inactive) of one or more nucleosomes on a promoter or enhancer region. Global chromatin structural changes have been shown to occur in the human α-globin gene locus by the action of the distal LCR (Forrester, W. C. et al., Genes Dev., 4, 1637 (1990)). In addition, active genes are characterized as containing hyperacetylated histones and undermethylated DNA. Interestingly, both global and local levels of chromatin structural perturbation often require the interaction of regulatory proteins with histone amino-terminal tails within the nucleosome, which are also the main targets of post-translational modification (for review, see Davie, J. R., Curr. Opin. Nucleic Genet. Dev., 8, 173 (1998)). Two critical pathways that facilitate this interaction involve distinct protein complexes that function either as motors to disrupt nucleosomes (ATP-driven chromatin remodeling complexes) or as enzymatic machinery to modify histones chemically and alter their affinity for DNA (histone acetylases, HATs/deacetylases, HDACs/kinases).

The mechanisms by which specific genes are activated in chromatin have been investigated in a variety of biochemical and genetic systems. Paranjape, S. M. et al., Annu. Rev. Biochem., 63, 265 (1994). Recent in vitro studies have focused on the role of specific cellular and viral factors in chromatin structural reconfiguration and nucleic acid expression. A common theme to emerge is that chromatin remodeling and transcriptional activation are separate processes that can be regulated by distinct proteins or subunits/domains of a given protein. This was shown originally with the GAL4-VP16 activator using chromatin-assembled genes. Pazin, M. J. et al, Science, 266, 2007 (1994). The observation that chromatin accessibility is not sufficient for transcription has important regulatory implications as nucleic acids can be preset by chromatin remodeling to be activated at a later time.

There are seven chromatin remodeling complexes that have been described to date: SWI/SNF, RSC, NURF, CHRAC, ACF, NURD and RSF. All are multi-subunit complexes with molecular weights ranging from 2 MDa to 0.5 MDa. Biochemical analyses have shown that these complexes can disrupt nucleosomal structure in a ATP-dependent manner (all complexes), facilitate factor binding (SWI/SNF, NURF, ACF) and transcription from chromatin-assembled genes (NURF, ACF and RSF). Several properties indicate that these complexes are functionally and mechanistically distinct. For example, RSC is an abundant complex in yeast and is encoded by essential genes, in marked contrast to SWI/SNF (SWI stands for mating type SWItch and SNF for Sucrose Non-Fermenting), suggesting a different biological role for these two complexes. Cairns, B. R. et al., Cell, 87, 1249 (1996). Furthermore, NURF has recently been shown to facilitate transcriptional activation from preformed chromatin templates in combination with GAL4-HSF. Mizuguchi, G. et al., Mol. Cell. 1, 141 (1998). In this assay, NURF cannot be replaced by either yeast SWI/SNF or CHRAC.

A novel complex has recently been described, using a purified in vitro transcription system, that alleviates the nucleosomal block to elongation. Orphanides, G. et al., Cell, 92, 105 (1998). This 230 kDa complex, called FACT (facilitates chromatin transcription), appears to function quite distinctly from chromatin remodeling complexes as it does not facilitate transcriptional initiation or require ATP hydrolysis. Thus, promoter-proximal chromatin remodeling is one critical step in gene activation but is not sufficient for transcription unless coupled with activities, such as FACT, which permit efficient elongation through nucleosomes.

Little is known about the manner in which remodeling complexes disrupt nucleosomes. Recent studies demonstrate that the ability of NURF to alter nucleosomal structure is impaired by crosslinking of nucleosomal histones, removal of amino-terminal histone tails, or mutation of lysine residues within the histone H4 tail; this indicates that the flexible tails play a critical role in the remodeling process. Georgel, P. T. et al., EMBO. J, 16, 4717 (1997). The formation of a ternary complex composed of DNA, histones, and activator is facilitated by SWI/SNF, which results in a destabilization, but not the loss of, nucleosomes that persists after the removal of SWI/SNF. Owen-Hughes, T. et al., Science, 273, 513 (1996). Recent studies demonstrate that the SWI/SNF-dependence of genes regulated by the yeast activator GAL4 is determined by the presence of low, rather than high, affinity GAL4 DNA binding sites on the promoter. The presence of high-affinity sites or a nucleosome-free region can overcome the requirement for this remodeling complex in vivo. Burns, L. G. et al., Mol. Cell. Biol., 17, 4811 (1997).

Interestingly, one subunit of NURF has recently been identified as the WD repeat protein, p55, which is also a subunit of Drosophila CAF1 (chromatin assembly factor 1). p55 homologs are found associated with histone acetyltransferases and deacetylases. Thus, many of the diverse chromatin-altering complexes may utilize common subunits. Martinez-Balbas, M. A. et al., Proc. Natl. Acad. Sci. USA, 95, 132 (1998).

It is clear that multiple levels of control are involved in regulated nucleic acid expression, from the activation of the chromosomal domain in which a nucleic acid resides to the formation of a basal initiation complex on a given promoter within the domain. Questions remain as to how tissue- or developmental-state-specific expression is established and how coordinate expression of multiple genes is achieved. In addition, the mechanism by which critical DNA control elements, often acting at long-range, such as enhancers, insulators, silencers, and LCRs, regulate transcription is still poorly understood.

### BRIEF SUMMARY

Provided herein are methods useful for modulating gene expression by regulating chromatin remodeling processes. Genes within chromatin DNA often are sequestered from the transcription factors that regulate their expression as the chromatin DNA often exists in a condensed state that renders the genes inaccessible to the transcription factors. The genes can become accessible to the transcription factors when the condensed chromatin relaxes and opens in a process referred to as “chromatin remodeling.” Chromatin remodeling is regulated in part by a protein complex composed of multiple protein subunits, and different chromatin remodeling complexes can be distinguished from one another by the different subunits within each complex. For example, certain chromatin remodeling complexes include a BRM ATPase and others include a BRG-1 ATPase. Transcription factors interact with chromatin remodeling complexes, and it has been discovered that certain transcription factors selectively interact with particular chromatin remodeling complexes, as described hereafter.

Thus, in one aspect, provided herein is a method for identifying a compound that modulates gene expression, which comprises (a) contacting (1) a SWI/SNF chromatin remodeling complex containing BRG-1 ATPase, or a portion of the remodeling complex, (2) a transcription factor containing a tryptophan cluster or a leucine zipper, or a fragment of the transcription factor, and (3) a test compound; and (b) determining whether there is an increase or decrease in the interaction between the chromatin remodeling complex or portion thereof and the transcription factor or fragment thereof in the presence and absence of the test compound, whereby a test compound that increases or decreases the interaction relative to the interaction in the absence of the test compound is identified as a compound that modulates gene expression. In one embodiment, step (a) further comprises contacting with a chromatin assembled DNA containing a gene of interest. The contacting in this method and other methods described herein may be carried out in any order.

Also provided herein is a method for identifying a compound that modulates gene expression, which comprises (a) contacting a chromatin assembled DNA containing a gene of interest with (1) a SWI/SNF chromatin remodeling complex containing BRG-1 ATPase, or a portion of the remodeling complex, (2) a transcription factor containing a tryptophan cluster or a leucine zipper, or a fragment of the transcription factor, and (3) a test compound; and (b) determining whether there is an increase or decrease in chromatin remodeling of the DNA containing the gene of interest in the presence and absence of the test compound, whereby a test compound that increases or decreases chromatin remodeling relative to the chromatin remodeling in the absence of the test compound is identified as a compound that modulates gene expression. In one embodiment, the increase or decrease in chromatin remodeling is determined by analyzing one or more DNaseI hypersensitive sites in the DNA containing the gene of interest.

Further provided herein is a method for identifying a compound that modulates gene expression, which comprises: (a) contacting a chromatin assembled DNA containing a gene of interest with (1) a SWI/SNF chromatin remodeling complex containing BRG-1 ATPase, or a portion of the remodeling complex, (2) a transcription factor containing a tryptophan cluster or a leucine zipper, or a fragment of the transcription factor, and (3) a test compound; and (b) determining whether there is an increase or decrease in transcription of the gene of interest in the presence and absence of the test compound, whereby a test compound that increases or decreases transcription relative to the transcription in the absence of the test compound is identified as a compound that modulates gene expression.

In one embodiment, the transcription factor contains a leucine zipper, or a fragment thereof. In a specific embodiment, the transcription factor is c-fos, c-jun, or C/EBPα, or a fragment thereof. In another specific embodiment, the transcription factor is phosphorylated CREB, or a fragment thereof and the gene of interest is a cAMP-responsive gene.

In another embodiment, the transcription factor contains a tryptophan cluster, or a fragment thereof. In a specific embodiment, the transcription factor is IRF-1, or a fragment thereof.

In one embodiment, the transcription factor fragment contains one or more regions selected from the group consisting of a leucine zipper, a tryptophan cluster, a DNA binding domain, an activation domain, a regulatory domain, a protein interaction domain, or a fragment of the foregoing.

Also provided herein is a method for identifying a compound that modulates gene expression, which comprises (a) contacting (1) a SWI/SNF chromatin remodeling complex containing BRG-1 ATPase, or a portion of the remodeling complex, (2) a transcription factor fragment consisting of a C4 motif or C2H2 motif from a zinc finger domain, and (3) a test compound; and (b) determining whether there is an increase or decrease in the interaction between the chromatin remodeling complex or portion thereof and the transcription factor fragment in the presence and absence of the test compound, whereby a test compound that increases or decreases the interaction relative to the interaction in the absence of the test compound is identified as a compound that modulates gene expression. In one embodiment, step (a) further comprises contacting with a chromatin assembled DNA containing a gene of interest.

Provided herein is a method for identifying a compound that modulates gene expression, which comprises (a) contacting a chromatin assembled DNA containing a gene of interest with (1) a SWI/SNF chromatin remodeling complex containing BRG-1 ATPase, or a portion of the remodeling complex, (2) a transcription factor fragment consisting of a C4 motif or C₂H₂ motif from a zinc finger domain, and (3) a test compound; and (b) determining whether there is an increase or decrease in chromatin remodeling of the DNA containing the gene of interest in the presence and absence of the test compound, whereby a test compound that increases or decreases chromatin remodeling relative to the chromatin remodeling in the absence of the test compound is identified as a compound that modulates gene expression. In one embodiment, the increase or decrease in chromatin remodeling is determined by analyzing one or more DNaseI hypersensitive sites in the DNA containing the gene of interest.

Further provided herein is a method for identifying a compound that modulates gene expression, which comprises (a) contacting a chromatin assembled DNA containing a gene of interest with (1) a SWI/SNF chromatin remodeling complex containing BRG-1 ATPase, or a portion of the remodeling complex, (2) a transcription factor fragment comprising a C4 motif or C₂H₂ motif from a zinc finger domain and an activation domain, and (3) a test compound; and (b) determining whether there is an increase or decrease in transcription of the gene of interest in the presence and absence of the test compound, whereby a test compound that increases or decreases transcription relative to the transcription in the absence of the test compound is identified as a compound that modulates gene expression.

In one embodiment, the transcription factor fragment contains one or more regions selected from the group consisting of a DNA binding domain, a regulatory domain, a protein interaction domain, or a fragment of the foregoing.

Provided herein is a method for identifying a compound that modulates gene expression, which comprises (a) contacting (1) a SWI/SNF chromatin remodeling complex containing BRM ATPase, or a portion of the remodeling complex, (2) a transcription factor or fragment thereof, and (3) a test compound; and (b) determining whether there is an increase or decrease in the interaction between the chromatin remodeling complex or portion thereof and the transcription factor or fragment thereof in the presence and absence of the test compound, whereby a test compound that increases or decreases the interaction relative to the interaction in the absence of the test compound is identified as a compound that modulates gene expression. In one embodiment, step (a) further comprises contacting with a chromatin assembled DNA containing a gene of interest.

Also provided herein is a method for identifying a compound that modulates gene expression, which comprises (a) contacting a chromatin assembled DNA containing a gene of interest with (1) a SWI/SNF chromatin remodeling complex containing BRM ATPase, or a portion of the remodeling complex, (2) a transcription factor or fragment thereof, and (3) a test compound; and (b) determining whether there is an increase or decrease in chromatin remodeling of the DNA containing the gene of interest in the presence and absence of the test compound, whereby a test compound that increases or decreases chromatin remodeling relative to the chromatin remodeling in the absence of the test compound is identified as a compound that modulates gene expression. In one embodiment, the increase or decrease in chromatin remodeling is determined by analyzing one or more DNaseI hypersensitive sites in the DNA containing the gene of interest.

Provided herein is a method for identifying a compound that modulates gene expression, which comprises (a) contacting a chromatin assembled DNA containing a gene of interest with (1) a SWI/SNF chromatin remodeling complex containing BRM ATPase, or a portion of the remodeling complex, (2) a transcription factor or fragment thereof, and (3) a test compound; and (b) determining whether there is an increase or decrease in transcription of the gene of interest in the presence and absence of the test compound, whereby a test compound that increases or decreases transcription relative to the transcription in the absence of the test compound is identified as a compound that modulates gene expression.

In one embodiment, the transcription factor contains an ankyrin repeat, or a transcription factor fragment thereof. In a specific embodiment, the transcription factor is ICD22 or CBF-1, or a fragment thereof. In a specific embodiment, the gene of interest is a Notch receptor regulated gene.

In one embodiment, the transcription factor fragment contains one or more regions selected from the group consisting of an ankyrin repeat, a DNA binding domain, an activation domain, a regulatory domain, a protein interaction domain, or a fragment of the foregoing.

Further provided herein are test compounds identified as modulating gene expression by one or more of the above methods.

Provided herein is a method for modulating gene expression, which comprises contacting a chromatin assembled DNA containing a gene of interest with (a) a transcription factor containing a tryptophan cluster or a leucine zipper, or a fragment of the transcription factor, and (b) a SWI/SNF chromatin remodeling complex containing BRG-1 ATPase, or a portion of the remodeling complex. In one embodiment, the transcription factor contains a leucine zipper, or fragment thereof. In a specific embodiment, the transcription factor is c-fos, c-jun, or C/EBPα, or a fragment thereof. In another specific embodiment, the transcription factor is phosphorylated CREB, or a fragment thereof. In one embodiment, the gene of interest is a cAMP-responsive gene. In another embodiment, the transcription factor contains a tryptophan cluster, or a fragment thereof. In a specific embodiment, the transcription factor is IRF-1, or a fragment thereof.

In one embodiment, the transcription factor fragment contains one or more regions selected from the group consisting of a leucine zipper, a tryptophan cluster, a DNA binding domain, an activation domain, a regulatory domain, a protein interaction domain, or a fragment of the foregoing.

Further provided herein is a method for modulating gene expression, which comprises contacting a chromatin assembled DNA containing a gene of interest with (a) a SWI/SNF chromatin remodeling complex; (b) a first transcription factor or fragment thereof comprising an activation domain, wherein the first transcription factor or fragment thereof does not interact with the SWI/SNF chromatin remodeling complex comprising BRG-1 ATPase to initiate transcription, and (c) a DNA-binding domain or fragment thereof of a second transcription factor, wherein the DNA-binding domain or fragment thereof of the second transcription factor interacts with the remodeling complex thereby allowing access of the first transcription factor or fragment thereof to the gene of interest, whereby gene expression is modulated. In one embodiment, the SWI/SNF chromatin remodeling complex comprises BRG-1 ATPase. In a specific embodiment, the DNA-binding domain of the second transcription factor contains a zinc finger, or a fragment thereof. In another embodiment, the SWI/SNF chromatin remodeling complex comprises BRM ATPase.

Also provided herein is a method for modulating gene expression, which comprises contacting a chromatin assembled DNA containing a gene of interest with (a) a transcription factor containing an ankyrin repeat, or a fragment of the transcription factor; and (b) a SWI/SNF chromatin remodeling complex containing BRM ATPase, or a portion of the remodeling complex. In one embodiment, the transcription factor is ICD22 or CBF-1, or a fragment thereof. In one embodiment, the gene of interest is a Notch receptor regulated gene.

Further provided herein are high-throughput screening assays that identify small molecule compounds that enhance or block the association between chromatin remodeling complexes and the specific transcription factors with which they interact, such as the BRG-1 subunits of the SWI/SNF complex and various transcription factors.

### BRIEF DESCRIPTION OF DRAWINGS

FIG. 1. A Zinc Finger DNA Binding Domain Directs SWI/SNF Remodeling to Enable Interaction by Proteins that Cannot Access their Sites in Chromatin. (A) In vitro transcription of chromatin-assembled HIV-1 promoters: effect of zinc finger DBD-targeted SWI/SNF remodeling on TFE-3 activation. 1 μg of chromatin was incubated in the presence or absence of hSWI/SNF and, where indicated, 50 pmol of TFE-3 and 125 pmol of the EKLF ZF DBD. All factors were added after chromatin assembly (post-assembly) except in lane 2. Reactions were split in half and analyzed separately by transcription and footprinting. α-globin plasmids were transcribed as internal controls. A diagram of the HIV-1 promoter and protein binding sites is depicted below. (B) DNase I footprint of chromatin-assembled HIV-1 promoters: effect of zinc finger DBD-targeted SWI/SNF remodeling on TFE-3 binding. Bars indicate binding sites for ZFP DBD (CACC site) and TFE-3. (C) Model showing how SWI/SNF-dependent structural remodeling by a zinc finger DBD facilitates binding and transcriptional activation by factors (TFE-3) that cannot access their sites in chromatin.

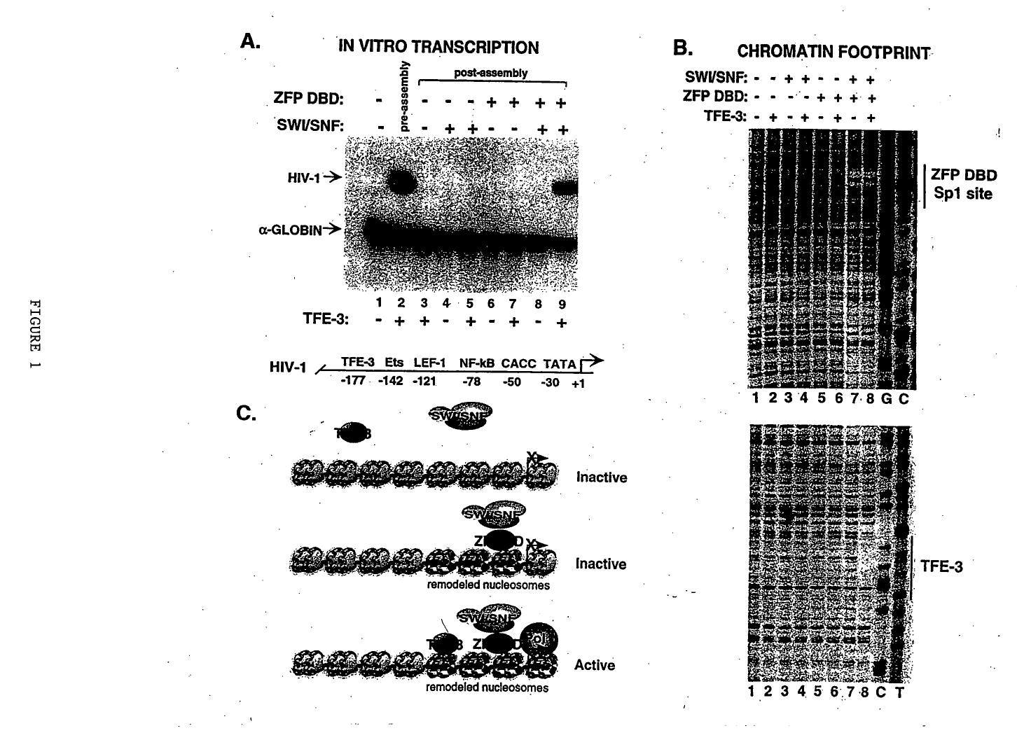

FIG. 2. SWI/SNF-dependent Remodeling and Transcription is Targeted by Individual Zinc Fingers within the C₂H₂ and C4 DNA Binding Domains. (A) Schematic representation of the zinc finger DBD structures of the KLF (C₂H₂) and GATA (C4) protein families. Cofactors that associate with each DBD or with individual ZFs are indicated. (B) DNase I hypersensitivity of chromatin-assembled β-globin promoters is generated by SWI/SNF and ZF DBDs from KLF, and Sp1, and individual GATA-1 ZFs. Assembled chromatin was incubated with hSWI/SNF and 35 pmol of each protein as shown before digestion with 2 and 1 U DNase1. Brackets indicate the −120 to +10 region of the promoter. A diagram of the β-globin promoter and protein binding sites is depicted below. (C) GST-pulldown analysis of protein interactions between 2 μg recombinant BRG-1 and 1 μg individual ZFs (F1, F2, F3) within the KLF ZF DBD. B=bound; S=supernatant. BRG-1 was detected by immunoblotting using BRG-1 antisera. (D) In vitro transcription of chromatin-assembled β-globin promoters: effect of individual KLF ZFs as dominant negative inhibitors of SWI/SNF-dependent EKLF activation. Assembled chromatin was incubated with the following proteins as indicated: WT-HIS, —HIS2=two preparations of histidine-tagged EKLF (37 pmol); F123=histidine-tagged EKLF DBD; GST F123=GST-tagged EKLF DBD; F1, F2, F3=individual KLF zinc fingers (250 pmol). AdLuc transcripts are shown as internal controls. In these experiments, ZF DBD or individual ZF were incubated with EKLF and SWI/SNF for 15 min on ice before addition to assembled chromatin. (E) DNase I footprint of chromatin-assembled β-globin promoters: effect of individual KLF ZFs as dominant negative inhibitors of SWI/SNF-dependent EKLF binding.

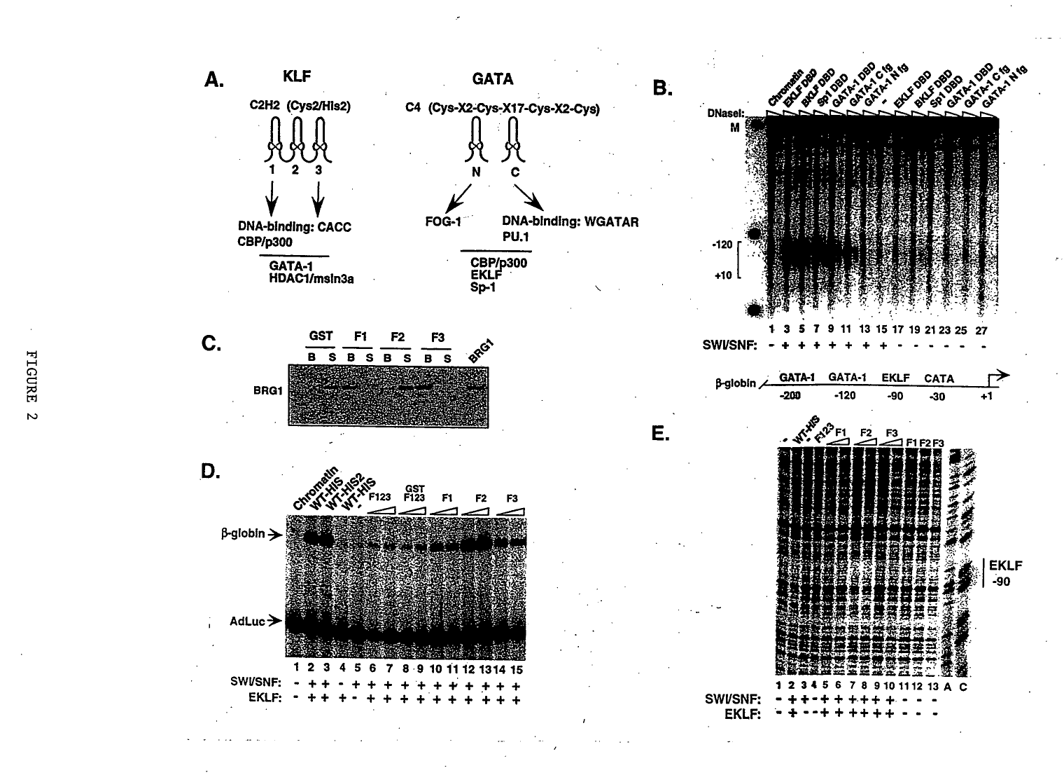

FIG. 3. Zinc Finger DNA Binding Proteins Specifically Interact with BRG-1-containing SWI/SNF Complexes. (A) SDS-PAGE silver stain analysis of epitope-tagged native hSWI/SNF complexes purified from HeLa INI11 cells (left panel). Immunoblot analysis of hSWI/SNF using antisera to BRG-1 and BRM subunits show the presence of both complexes in our preparations (right panel). (B) GST-pulldown analysis of protein interactions between 3 μg native hSWI/SNF and 1 μg recombinant ZF DBDs of EKLF, BKLF, Sp1, retinoic acid receptors (RAR and RXR), GATA-1, and individual GATA-1 C— and N-ZFs. Interactions with BRG-1 or BRM were detected by immunoblotting using appropriate antisera. (C) GST-pulldown analysis of protein interactions between 500 ng recombinant BRG-1 (upper panel) or BRM (lower panel) and 500 ng recombinant ZF DBDs, individual ZFs, ICD22 and CBF-1. 100% of bound proteins (beads), 50% of unbound proteins (sup), and 50% of input BRG-1 and BRM were analyzed on 10% SDS-PAGE gels and immunoblotted with antibodies against BRG-1 or BRM. (D) In vivo interaction of EKLF with SWI/SNF. Co-immunoprecipitation of EKLF with antisera to BRG-1, BRM, BAF155 and BAF170 subunits of SWVSNF using 100 μg mouse erythroid (MEL) cell nuclear extract. Immune complexes were separated by 10% SDS-PAGE and immunoblotted with antibodies against EKLF. Recombinant EKLF (25 ng) was used as a positive control (lane 7).

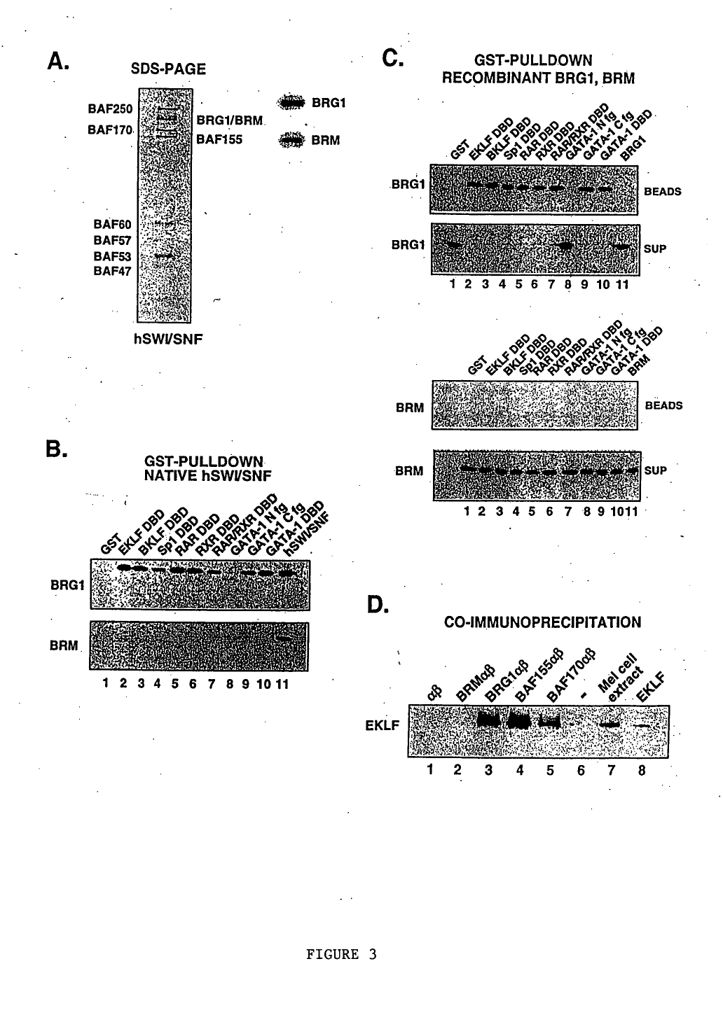

FIG. 4. Native and Recombinant BRG-1 but not BRM SWI/SNF Complexes Coactivate Transcription by Zinc Finger Proteins. (A) EKLF-dependent transcription from chromatin-assembled β-globin promoters with native INI-1 tagged SWI/SNF (containing both BRG-1 and BRM), immunopurified native BRG-1 or BRM SWI/SNF, and recombinant BRG-1 or BRM complexes. 100 ng of chromatin were incubated with 3.7 pmol EKLF, 20 ng F-BRG-1, 100 ng F-BRM, 100 ng of F-BAF155, 58 ng INI-1 tagged SWI/SNF, 60 ng or 120 ng immunopurified BRG-1- or BRM-containing SWI/SNF as indicated. Two separate experiments are represented in the right and left panels. AdLuc transcripts are shown as internal controls. A diagram of the β-globin promoter is depicted below. (B) Nucleosome disruption analysis showing that the recombinant BRM ATPase possesses classical chromatin remodeling activity. 300 ng of SWI/SNF (lanes 4-5), 100 ng (lanes 6-7) and 500 ng (lanes 8-9) of hBRM were incubated with reconstituted 5S mononucleosomes followed by DNase 1 digestion. M=MspI digested pBR322 as a molecular weight marker. (C) Equal amounts (100 ng) of native INI-1 tagged SWI/SNF and immunopurified BRG-1 or BRM SWVSNF complexes were analyzed by Western blotting using antisera to BRG-1, BRM, BAF 155 and INI-1. (D) ATPase activity of SWI/SNF complexes. Approximately 55 ng of native SWI/SNF, immunopurified BRG-1 and BRM SWI/SNF complexes were incubated in the presence or absence of 150 nM nucleosomes and (γ-³²P) ATP for the indicated times. The phosphate present at time zero is due to the presence of (γ-³²P) phosphate in the (γ-³²P) ATP stock. The ratio of inorganic phosphate to ATP was quantitated for each time point using a Molecular Dynamics Phospholmager. (E) Chromatin structural analysis of in vitro assembled β-globin plasmids by micrococcal nuclease digestion.

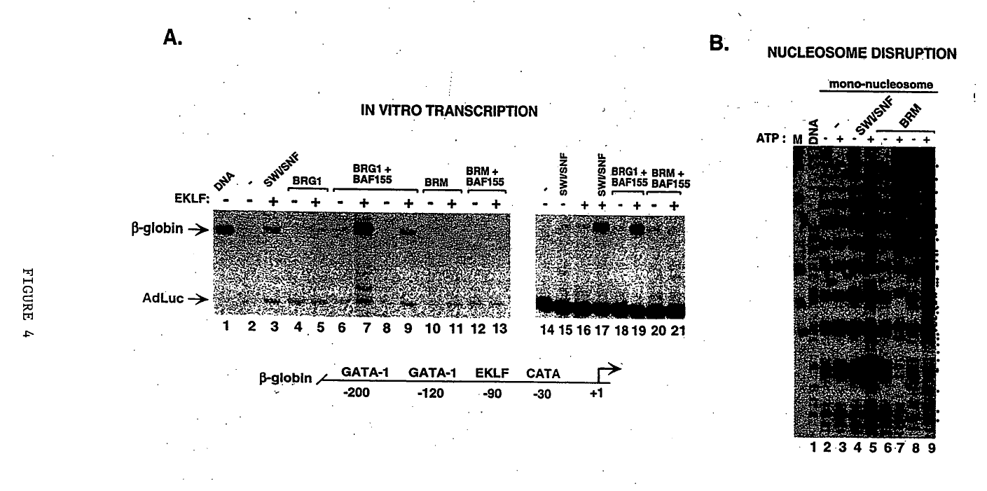

FIG. 5. BRG-1 Interacts with EKLF through Unique N-terminal Sequences which are Nonhomologous in BRM to Target Remodeling and Activate Transcription (A) Diagram of human BRM and BRG-1 proteins comparing common (HPV E7, KR, Bromodomain) and unique (99 bp exon, N-terminus) motifs between the ATPases. The hatched area near the BRM N-terminus designates a region that is nonhomologous with BRG-1. Cloned subdomains of BRG-1 containing common and unique sequences with BRM are indicated below. (B) GST-pulldown analysis of protein interactions between 350 ng of his-tagged EKLF and 500 ng of each BRG-1 subdomain. Interactions were detected by immunoblotting using EKLF antisera. Purified EKLF was used as a positive control (lane 1). (C) In vitro transcription of chromatin-assembled β-globin promoters: effect of recombinant BRG-1 subdomains as dominant negative inhibitors of SWI/SNF-dependent EKLF activation. Assembled chromatin templates were incubated with EKLF (37 pmol per 1 μg of chromatin in a 100 μl reaction volume), SWI/SNF, and 300 pmol of each BRG-1 subdomain. Reactions were divided in half and used for transcription or DNase hypersensitivity. AdLuc transcripts are shown as internal controls. (D) DNase I footprint of chromatin-assembled β-globin promoters: effect of recombinant BRG-1 subdomains as dominant negative inhibitors of SWI/SNF-dependent EKLF binding. Each chromatin reaction (see above) was again divided into 2 tubes of 150 ng and digested with 1 and 2 U DNase1.

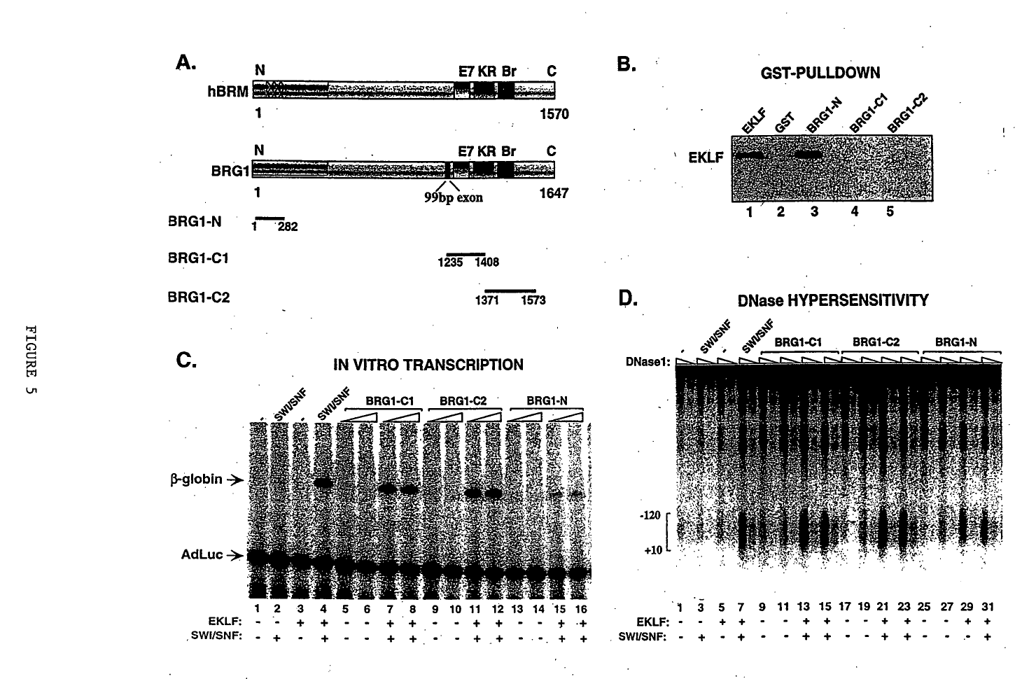

FIG. 6. BRG-1 and BRM Possess Distinct Interaction Specificities towards Different Transcription Factors. (A) In vitro binding studies between 500 ng native hSWI/SNF and 200 ng of the following in vitro translated proteins: c-fos, c-jun, heterodimer of c-fos/c-jun, NF-κB p50 DBD subunit, LEF HMG DBD, C/EBPα, IRF-1, and EKLF DBD. Immunoprecipitation was carried out using antisera to BRG-1, BRM and INI1 subunits of the hSWI/SNF complex and analyzed by SDS-PAGE. 10% of the in vitro translated protein was loaded as an input. (B) GST-pulldown analysis of interactions between 1 μg hSWI/SNF and 500 ng of the following GST-fused proteins: EKLF DBD, TFE-3, ICAT, β-catenin, ICD22, and CBF-1. Interactions were detected by immunoblotting using antibodies against SWI/SNF BRG-1, BRM, and INI1 subunits. Purified hSWI/SNF was used as a positive control. (C) In vivo interaction of CBF-1 with BRM-containing SWI/SNF. Co-immunoprecipitation of hSWI/SNF with antibodies to CBF-1 using whole cell extracts from Notch2-expressing mouse myoblasts (C2C12) and control cells. Immune complexes were separated by SDS-PAGE and analyzed by Western blots with antisera against BRG-1, BRM, and INI1 subunits of SWI/SNF. Native hSWI/SNF was used as a positive control (lane 7).

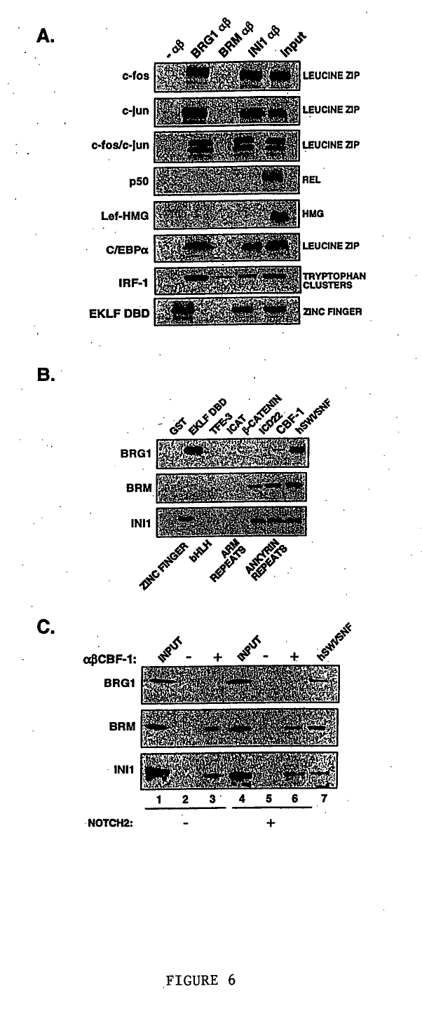

FIG. 7. Promoter-Specific Recruitment of BRG-1 or BRM SWI/SNF and Chromatin Modifiers during Proliferation, Differentiation, and Signal Transduction. Chromatin immunoprecipitation (ChIPs) analyses of SWI/SNF remodeling complexes (BRG-1, BRM, core subunit INI1), transcriptional activators (EKLF/Sp1, CBF-1, Notch2), chromatin coactivators/corepressors (HDACI, CBP), and histone modifications (ACH3, ACH4, dimethyl lys4H3, core H3) were performed using a variety of promoters (left side) which are up- or downregulated or permanently inactivated (right side, indicated by arrows or by “X”) in the cell types examined. Briefly, formaldehyde crosslinked DNA was immunoprecipitated, using the antibodies indicated above each lane and the recovered DNA was employed as a template in PCR amplification using primers directed against different promoters. (A) ChIP analyses of individual promoters during DMSO-induced mouse erythroid cell differentiation (βmaj=adult β-globin; DHFR=dihydrofolate reductase; TCRα=T cell receptor alpha chain). Amplification products were in the range of 250 to 350 bp and contain the promoter sequence elements indicated on the left of the panel. Input DNA (5%) and minus antibody controls were also included. (B) ChIP analyses of Hes1 (hairy enhancer of split) and Hes5 promoters during Notch signaling in mouse myoblasts. Amplification products were in the range of ˜400 bp and contain the CBF-1 sequence elements indicated on the left of the panel. Input DNA (5%) and minus antibody controls were also included.

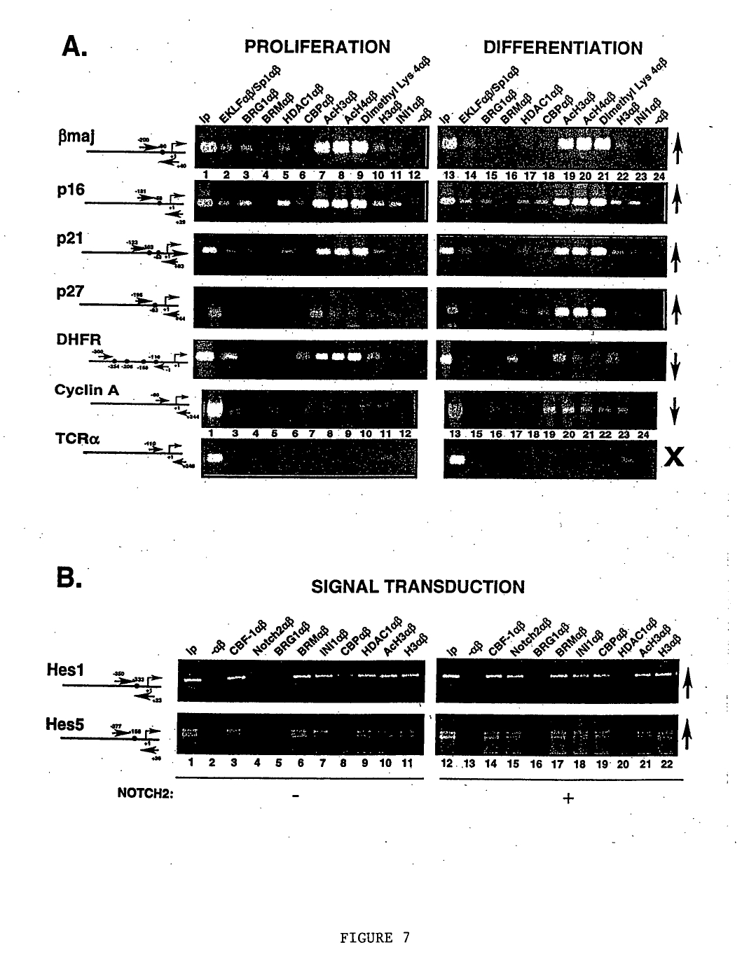

FIG. 8. To determine the ability of SWI/SNF to interact with phosphorylated CREB, GST pull-down assays were performed with 3 μg human SWI/SNF and 1 μg GST-fused Phosphorylated and unphosphorylated CREB. The SWI/SNF BRG-1 subunit was then detected by Western blot analyses using the antisera at 1:1000 dilution. EKLF DBD and phosphorylated CREB bound SWI/SNF BRG-1 subunit while no binding was observed with unphosphorylated CREB. SWI/SNF BRM subunit did not bind EKLF DBD, phosphorylated CREB or unphosphorylated CREB.

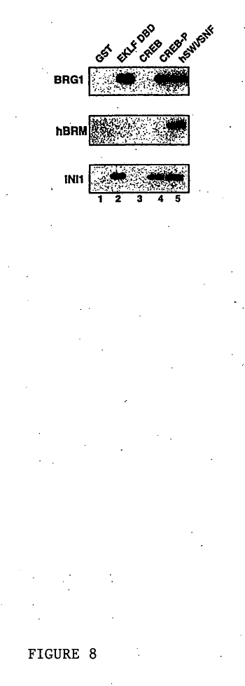

### DETAILED DESCRIPTION

The compositions and methods provided herein create accessible chromatin structures and can be used to identify compounds that modulate chromatin remodeling of specific DNA sequences. Chromatin structure acts as a primary regulator of gene expression by controlling access to the regulatory DNA sequences of a gene. More specifically, a gene in a condensed chromatin is inaccessible to factors that bind its regulatory DNA sequences and thus is inactive. On the other hand, the regulatory DNA sequences in a gene in remodeled chromatin (i.e., that is loose and extended with greater nucleosome mobility) is accessible to interact with the factors that control gene expression. The factors binding regulatory DNA sequences are collectively known as transcription factors and act as a secondary regulator of gene expression. For example, a typical gene is controlled by a promoter region located just ahead of the beginning of the coding sequence of the gene. Transcription factors (such as activators, RNA polymerase, etc.) interact with the nucleic acid promoter to initiate transcription of the gene. Nucleosomes (or chromatin) impose a physical barrier to the interaction between transcription factors and regulatory DNA sequences. If these factors cannot interact with the nucleic acid regulatory region, such as a DNA promoter region, then the nucleic acid cannot be expressed. When nucleosomal structure is remodeled to loosen its affinity for the nucleic acid (i.e., creating greater mobility), transcription factors can interact with the promoter and activate gene expression. The ability of chromatin to control gene expression in this manner is fundamental in the development and long term health of all organisms in permitting the establishment and maintenance of distinct cell types with specialized physiological functions in coherent organ systems.

The methods described herein are useful for identifying compounds that modulate protein-protein interactions, chromatin remodeling, and gene transcription and expression. Such compounds can be formulated as compositions and administered therapeutically. The methods described herein also are useful in identifying transcription factors that interact with chromatin remodeling complexes.

The compositions and methods provided herein are useful in the treatment of symptoms and diseases where transcription factor dysfunction or non-function is implicated. For example, transcription factors containing zinc finger DNA-binding domains have been implicated in human disease. See, e.g., ZINC-FINGER PROTEINS IN ONCOGENESIS: DNA-BINDING AND GENE REGULATION (M. Sluyser, ed. Annals of the New York Academy of Sciences 684, 1993); GREGG L. SEMENZA, TRANSCRIPTION FACTORS AND HUMAN DISEASE (Oxford Monographs on Medical Genetics 37, 1998); DAVID S. LATCHMAN, EUKARYOTIC TRANSCRIPTION FACTORS (3d ed. 1999); Klug A., J. Mol. Biol. 293: 215-8 (1999); Thiel G, et al., Naturwissenschafte. 86: 1-7 (1999); Ashraf, et al., Curr Biol. 8:R683-6 (1998); Bieker J J, et al., Ann N Y Acad Sci. 850: 64-9 (1998); Takatsuji H., Plant Mol. Biol. 39: 1073-8 (1999); Chandrasegaran S, et al., Biol. Chem. 380: 841-8 (1999).

The compositions and methods provided herein can also be useful in the treatment of symptoms and diseases where chromatin remodeling's effect on gene expression is implicated. In one example, the absence of a particular chromatin remodeling complex, i.e., BRM containing chromatin remodeling complex, in mice results in obesity. Reyes, J. C., et al., EMBO J. 17: 6979-91 (1998). Because BRM knockout mice are obese, BRM complexes might be targeted to genes that control obesity or potentially diabetes. If so, then proteins that direct BRM to these genes could be identified. Also, BRM may be mutated in diabetes or obesity and its function could be restored therapeutically. BRM assays and future target gene and interacting protein identification will be valuable as drug screening targets and for diagnostics.

Additionally, the compositions and methods identified herein can modulate hematopoietic stem cell renewal and the restriction of cell fates (e.g., differentiation decisions). In particular, Notch-mediated cellular interactions are known to regulate cell fate decisions in a variety of developmental systems. Ohishi, K. et al., Int. J. Hematol. 75: 449-59 (2002). Therefore, the preferential interaction of BRM-ATPase with genes in the Notch signaling pathway offers a unique opportunity to identify compounds that can modulate cell fate decisions. For example, the Notch ligand, Delta 1, appears to complement cytokine mediated cell fate decisions. Vamum-Finney, B., et al., Blood 101: 1784-89 (2003). While in another study, Notch signaling appeared to play a decisive role in macrophage and dendritic cell differentiation. Ohishi, K., et al., Blood 98: 1402-07 (2001). Furthermore, constitutive Notch 1 signaling permitted immortalization of cytokine-independent hematopoietic stem cells that retained the ability to generate progeny with lymphoid and myeloid characteristics, implicating Notch signaling in stem cell renewal. Varnum-Finney, B., et al., Nature Med. 6: 1278-81 (2000).

Chromatin, Chromatin Remodeling Complexes, and Regulatory Factors

Chromatin is a network of DNA and associated proteins, primarily histones, and is either condensed or loosely decondensed. The fundamental unit of chromatin is the nucleosome, composed of DNA and histones. Chromatin structure can be altered through remodeling of the nucleosomal structure by chromatin remodeling complexes. Such remodeling is an ATP-dependent process that can alter nucleosome mobility and increase the access of DNA binding factors to the DNA sequence of the nucleosome, thereby creating a “loose, decondensed” nucleosomal structure. This structure facilitates the interaction of transcription factors with nucleic acid and result in gene activation. Therefore, targeted chromatin remodeling is an important step in gene activation that precedes transcription. These chromatin remodeling complexes are found in a wide variety of life forms, including both plants and animals.

Any chromatin assembled DNA can be used with the present methods. As used herein, the term “chromatin assembled DNA” refers to a complex of DNA and histones, and optionally non-histone proteins, that is condensed. In one embodiment, chromatin can be isolated as polynucleosomes from mammalian tissue culture cells using methods well known in the art. See, e.g., 4 CURRENT PROTOCOLS IN MOLECULAR BIOLOGY 21.5 (F. M. Ausubel, et al., eds. 2000). In another embodiment, chromatin can be assembled in vitro using a defined sequence of DNA encoding a gene of interest using a salt dialysis or an enzymatic chromatin assembly system. See, e.g., 4 CURRENT PROTOCOLS IN MOLECULAR BIOLOGY 21.6-21.7. Any source of histones can be employed for in vitro assembly of chromatin including, but not limited to histones from bacterial, insect, and mammalian sources. Any source of DNA template can be used for chromatin assembly including but not limited to bacterial, insect, and mammalian sources. In one embodiment, the source of histones can be restricted to the same organism, tissue, and cell-type as the DNA template, the chromatin remodeling complex, or both. Any enzymatic chromatin assembly system can be used. The components of such an enzymatic system can be derived from cell extracts or can be purified recombinant assembly factors. In one embodiment, chromatin is assembled in vitro using DNA constructs containing a gene of interest purified from a mammalian cell, human histones, and Drosophila embryonic extracts. See, e.g., J. A. Armstrong, et al., Cell 95:93-104 (1998); S. Kadam, et al., Genes Dev. 14: 2441-51 (2000). In one embodiment, the construct containing the human beta-globin gene as gene of interest. The construct is purified from transfected mouse erythroid leukemia (MEL) cells (American Tissue Cell Culture). In another embodiment, the construct containing the gene of interest is purified from mouse myoblasts expressing Notch2 ICD (C2C12). Nofziger, et al., Develop. 126: 1689 (1999).

Any chromatin remodeling complex can be used with the present methods. As used herein, the term “chromatin remodeling complex” refers to a complex of individual protein subunits that act in concert to mediate chromatin condensation or decondensation. Chromatin remodeling complexes useful in the present invention can include, but are not limited to SWI/SNF, RSC, NURF, CHRAC, ACF, NURD, and RSF. In one embodiment, the chromatin remodeling complex is SWI/SNF. Any SWI/SNF complex can be used in the methods herein. Mammalian SWI/SNF complexes consists of approximately 15 subunits and fall into two broad classes depending on the ATPase subunit the complex contains (i.e., BRG-1 ATPase or BRM ATPase). Kadam, et al., Genes Dev. 14: 2441-51 (2000). In one embodiment, the SWI/SNF complex comprises a complex of recombinant subunits, generated using methods well-known in the art. In another embodiment, the SWI/SNF complex is purified from nuclear extracts. The nuclear extracts can be derived from bacterial, insect, or mammalian cells. In one embodiment, the source of the SWI/SNF complex is mammalian. In a specific embodiment, the SWI/SNF complex is human. In one embodiment, the chromatin remodeling complex is SWI/SNF comprising BRG-1 ATPase. In another embodiment, the chromatin remodeling complex is SWI/SNF comprising BRM ATPase.

Any portion of the chromatin remodeling complex can be used in the methods provided herein. As used herein, the term “portion” refers to one or more subunits that associates with other subunits to form the chromatin remodeling complex, or fragments, analogues, or derivatives thereof. Portions of the chromatin remodeling complex may be used alone or in combination with other portions of the chromatin remodeling complex. The portion may be active alone or in combination with one or more other portions, fragments, analogs or derivatives thereof. The portions can be derived from nuclear extracts or can be recombinantly produced. Any nuclear extract can be used for chromatin remodeling complex portions including, but not limited to bacterial, insect, or mammalian cells. In one embodiment, the source of the subunit is mammalian. In a specific embodiment, the source of the portion is human. Exemplary portions include, but are not limited to BRG-1 ATPase, BRM ATPase, BAF 155, BAF 170, INi1, BAF 60, BAF 47, and BAF 57. In one embodiment, a minimal SWI/SNF complex is BRG-1 ATPase and BAF 155. In another embodiment, a minimal SWI/SNF complex is BRM ATPase and BAF 155. The methods provided herein are also useful in identifying other minimal chromatin remodeling complexes. Such portions can be evaluated for biological activity using the methods provided herein as indicators of interaction activity, remodeling activity, and transcriptional activity.

Any fragment of the chromatin remodeling complex portion can be used in the methods herein. As used herein, the term “fragment” refers to any biologically active fragment. The fragment may be active alone or in combination with one or more other portions, fragments, analogs or derivatives thereof. Such fragments can include only a part of the full-length sequence and yet possess the same function, possibly to a greater or lesser extent. For example, deletion mutants of a chromatin remodeling complex subunit can be designed and expressed by well known laboratory methods. Such fragments can be evaluated for biological activity using the methods provided herein as indicators of subunit interaction activity, remodeling activity, and transcriptional activity. As used herein, the term “region” refers to only a portion of the full-length sequence of a fragment or factor and is typically identified as encoding one or more amino acid motifs. The individual amino acid motifs may originate from the same or different full-length transcription factors. In one embodiment, the fragment is the N-terminus of the BRG-1 ATPase, typically amino acids 1-282 of the BRG-1 ATPase.

Any analog or derivative of the chromatin remodeling complex can be used in the methods herein. As used herein, the term “analog or derivative” refers to substituted and/or mutated proteins characterized by the ability to mediate chromatin remodeling alone or in concert with other subunits, subunit fragments, or subunit analogs or derivatives. Such mutations and substitutions can be designed and expressed by well-known laboratory methods. Such analogs or derivatives can be evaluated for biological activity using the methods provided herein as an indicator of protein-protein interaction activity, remodeling activity, and transcriptional activity.

Any transcription factor can be used with the methods provided herein. As used herein, the term “transcription factor” refers to any protein, other than RNA polymerase, required to initiate or regulate transcription. A transcription factor includes general factors required for the transcription of all genes (i.e., participate in formation of the transcription initiation complex near the start site) and specific factors that stimulate or repress transcription of particular genes by binding to specific regulatory sequences (e.g., enhancers, promoter-proximal elements). See MOLECULAR CELL BIOLOGY G-17 (H. Lodish, et al. eds., 3d ed. 1995). Transcription factors include, but are not limited to factors acting as activators, repressors, silencers, or modulators (e.g., enhancers) of transcription. Any source of transcription factors can be used for this invention. The transcription factor can be from a bacterial, insect, or mammalian cell. A transcription factor can also include mutants of naturally occurring transcription factors (e.g., having addition, deletions or different amino acid sequences). For example, deletion mutants of a transcription factor can be designed and expressed by well known laboratory methods. In one embodiment, the transcription factor is generated using well known molecular techniques. In another embodiment, the transcription factor is isolated from nuclear extracts of cell lines or tissue samples. Any description of transcription factor herein also includes a fragment, analog, or derivative of a transcription factor.

Transcription factors useful in the present invention with the SWI/SNF complex comprising BRG-1 ATPase can have any type of DNA-binding domain of a transcription factor identified by the methods provided herein, including but not limited to domains with a zinc finger motif, a leucine zipper motif, and a tryptophan cluster motif as described hereafter. Any of these transcription factors or fragments thereof can be used in any method described herein, such as methods that detect protein-protein interactions, chromatin remodeling, and gene transcription.

In one embodiment, the transcription factor comprises a zinc finger motif. As used herein, a “zinc finger motif” refers to a repeating motif that permits a region of a protein to fold around a central Zn²⁺ ion. In one embodiment, the zinc finger motif comprises a C₂H₂ motif. As used herein, a “C₂H₂ motif” refers to the sequence of the repeating unit within the zinc finger, where the sequence is (Tyr/Phe) X Cys X₂₋₄ Cys X₃ (Phe/Tyr) X₅ Leu X₂ His X₃₋₄ His, where X is any amino acid. See MOLECULAR CELL BIOLOGY 449. In another embodiment, the zinc finger motif comprises a C4 motif. As used herein, a “C4 motif” refers to the sequence of the repeating unit within the zinc finger, where the sequence is Cys X₂ Cys X₁₃ Cys X₂ Cys X₁₄₋₁₅ Cys X₅ Cys X₉ Cys X₂ Cys, where X is any amino acid. See MOLECULAR CELL BIOLOGY 449. Exemplary zinc finger proteins include, but are not limited to members of the KLF, Sp1, nuclear hormone receptor, and GATA protein families. In one embodiment, the transcription factor comprising a zinc finger motif includes, but is not limited to GATA-1 (erythroid), Sp1 (ubiquitous), EKLF (erythroid), FKLF (fetal), BKLF (basic), GKLF (gut), and LKLF (lung). In another specific embodiment, the zinc finger motif protein is a zinc finger motif-containing nuclear hormone receptors. Such receptors include, but are not limited to androgen, estrogen, thyroid, progesterone, and glucocorticoid receptors. In one embodiment, the zinc finger-containing nuclear hormone receptor is RAR and RXR. In a specific embodiment, the zinc finger-containing protein is Wilm's tumor suppressor protein (i.e., WT1) implicated in kidney differentiation and tumorigenesis. WT1 strongly regulates amphiregulin, a member of the epidermal growth factor family, among other genes. In a specific embodiment, the zinc finger proteins are the BRCA1 and BRCA2 proteins implicated in hereditary breast and ovarian cancers. In a specific embodiment, the zinc finger protein is KRAB repressor domain-containing proteins that are involved in epigenetic silencing of genes. In a specific embodiment, the zinc finger protein contain the BTB/POZ domain and includes, but is not limited to the PLZF (promyelocytic leukemia zinc finger) protein, which is fused to RARα (retinoic acid receptor alpha) in a subset of acute promyelocytic leukemias (APLs) and acts as a potent oncogene.

In another embodiment, the transcription factor comprises a leucine zipper motif. As used herein, a “leucine zipper motif” refers to a sequence within the DNA binding domain containing leucine residues present with regular, seven amino acid periodicity at every second turn along the hydrophobic face of an α-helix, where the amino acid sequence is[KR]-x(1,3)[RKSAQ]-N-x(2)[SAQ](2)-x[RKTAENQ]-x-R-x[RK]. Sauer, R. T. Nature 347: 514-15 (1990). In one embodiment, the transcription factor containing a leucine zipper is c-fos, c-jun, or C/EBPα. In another embodiment, the transcription factor is phosphorylated CREB.

In another embodiment, the transcription factor comprises a tryptophan cluster. As used herein, a “tryptophan cluster” refers to a sequence within the DNA binding domain containing several tryptophan residues, sometimes five tryptophan residues. For example, the tryptophan clusters sometimes comprise several tryptophan residues with a spacing of 12-21 amino acid residues. The subclass of myb-type DNA-binding domains typically exhibit a spacing of 19-21 amino acid residues. In one embodiment, the transcription factor containing a tryptophan cluster is a member of the family of Myb, Ets, or interferon regulating factors. In a specific embodiment, the transcription factor is IRF-1.

Transcription factors useful in the present invention with the SWI/SNF complex comprising BRM ATPase can have any type of DNA-binding domain, including but not limited to domains comprising an ankyrin repeat. As used herein, the term “ankyrin repeat” refers to tandemly repeated modules of about 33 amino acids. Any of these transcription factors or fragments thereof can be used in any method described herein, such as methods that detect protein-protein interactions, chromatin remodeling, and gene transcription. In one embodiment, the transcription factor is a member of the Notch signaling pathway. In another embodiment, the transcription factor is ICD22 or CBF-1, transcription factors in Notch family pathway. These proteins, ICD22 and CBF-1, are critical regulators of the Notch signaling pathway and function with the cofactor Mastermind to activate transcription of chromatin-assembled templates (Fryer, C. J., et al., Genes Dev. 16:1397-1411 (2002)).

Any transcription factor fragment can be used with the methods herein. As used herein, the term “fragment” refers to any biologically active fragment. The fragment may be active alone or in combination with one or more portions of the chromatin remodeling complex. The fragment can include one or more regions of a DNA binding domain, an activation domain, a regulatory domain, an interaction domain, or any fragment thereof. As used herein, the term “region” refers to only a portion of the full-length sequence of a fragment or factor and is typically identified as encoding one or more amino acid motifs, including but not limited to a zinc finger, a leucine zipper, a tryptophan cluster, and an ankyrin repeat. Such fragments can include only a portion of the full-length sequence and yet possess the same function, possibly to a greater or lesser extent. Fragments can include any number of amino acids. For example, a zinc finger fragment may be 26 or fewer amino acids for the C₂H₂ motif and 56 or fewer for the C4 motif zinc finger, with any zinc finger domain being useful in the methods herein. A transcription factor fragment can also include mutants of naturally occurring transcription factor fragments (e.g., having additions, deletions or different amino acids relative to the naturally occurring sequence). For example, deletion mutants of a transcription factor fragment can be designed and expressed by well known laboratory methods. Such fragments can be evaluated for modulating gene expression using the methods provided herein as indicators of subunit interaction activity, remodeling activity, and transcriptional activity.

Any analog or derivative of the transcription factor can be used in the methods herein. As used herein, the term “analog or derivative” refers to substituted and/or mutated proteins characterized by the ability to initiate transcription alone or in concert with other portions, fragments, analogs or derivatives thereof. Such mutations and substitutions can be designed and expressed by well-known laboratory methods. Such analogs or derivatives can be evaluated for biological activity using the methods provided herein as an indicator of protein interaction activity, remodeling activity, and transcriptional activity.

Throughout this specification, description pertaining to transcription factors is also applicable to transcription factor fragments, analogs or derivatives thereof.

Any DNA binding domain or fragment thereof can be used with the methods herein. As used herein, the term “DNA binding domain” refers to any domain of a transcription factor that binds DNA. The DNA binding domain may be active alone in binding the DNA or in combination with one or more portions of the chromatin remodeling complex. Such DNA binding domains can include only a portion of the full-length sequence and yet possess the same function, possibly to a greater or lesser extent. For example, deletion mutants of a DNA binding domain can be designed and expressed by well known laboratory methods. Such DNA binding domains can be evaluated for modulating gene expression using the methods provided herein as indicator of protein interaction activity, remodeling activity, and transcriptional activity. In one embodiment, the DNA binding domain or fragment thereof is a zinc finger binding domain. In a specific embodiment, the zinc finger binding domain is from the erythroid factor EKLF.

Any gene of interest may be used in the methods provided herein. As used herein, the term “gene of interest” refers to a DNA sequence that encodes at least a portion of the regulatory sequence for a target gene. The DNA sequence can include sequence encoding the full length gene product or some fragment thereof. The DNA sequence can include the regulatory and encoding sequences of more than one gene or some portion thereof. Any source of the gene of interest is useful with the present methods. In one embodiment, the gene of interest is a recombinant construct purified from a bacterial or mammalian cell line. In another embodiment, the gene of interest is isolated from a nuclear extract of cells or tissue sample. In one embodiment, the gene of interest is a cAMP-responsive gene where the transcription factor or transcription factor fragment is phosphorylated CREB. In another embodiment, the gene of interest is a Notch receptor regulated gene where the transcription factor or transcription factor fragment is ICD22 or CBF-1.

Detection of Modulation of Gene Expression

Modulation of gene expression that results from chromatin remodeling can be detected by the methods provided herein. The modulation of gene expression detected by the methods provided herein occurs through a number of discrete steps. First, the chromatin remodeling complex interacts and a transcription factor or transcription factor fragment that targets the complex to specific nucleosomal sites within the chromatin-assembled DNA for stable remodeling (i.e., the regulatory sequences of the gene of interest). Therefore, the modulation of gene expression can be detected by examining the interaction between one or more subunits of the chromatin remodeling complex and the transcription factor or transcription factor fragment. Second, the chromatin remodeling complex remodels (i.e., decondenses or loosens) the nucleosome structure to make the gene available to the transcriptional machinery. Because chromatin must first be remodeled prior to the active transcription of a gene of interest, the relative levels of chromatin remodeling also reflect the ability to modulate gene expression. Finally, the gene in the decondensed chromatin is transcribed. Hence, the increase or decrease in the amount of transcription reflects the modulation of gene expression by chromatin remodeling.

Any convenient method can be used to quantitate the interaction between one or more subunits of a chromatin remodeling complex and a transcription factor or transcription factor fragment. The proteins can be contacted in vitro or in vivo by any convenient protocol. The proteins can be contacted in any order or simultaneously in the presence or absence of DNA. The protein-protein interaction can be determined in the absence or presence of chromatin or chromatin assembled DNA. Chromatin assembled DNA can be isolated from nuclear extracts or assembled in vitro. For example, nuclei are released from cultured cells by physical shear forces (e.g., homogenization) and nonionic detergents (e.g., NP-40). The homogenate is then washed several times with a buffer (e.g., 0.4 M NaCl) to remove nonhistone proteins. The pellet is then solubilized at a suitable salt concentration (e.g., 0.65 M NaCl). Because the linker histones are released from the nuclei, the chromatin is decompacted, allowing it to be separated from other nuclear fragments by further homogenization. The homogenate is then dialyzed to reduce the salt concentration and digested with endonuclease micrococcal nuclease to generate smaller chromatin fragments. The chromatin can then be isolated by size using gradient centrifugation or gel filtration. Chromatin can be assembled in vitro using Drosophila embryonic extracts. For example, cell extracts are prepared from S-190 Drosophila embyros. The extracts are prepared using physical shear force (e.g., homogenization) to release the cell contents. Once homogenized, the aqueous supernatant is recovered after centrifugation and buffered (e.g., 1 M MgCl2). Core histones are prepare using micrococcal digestion of isolated nuclei followed by gradient centrifugation or hydroxylapatite chromatography.

In one embodiment, the interactions between the chromatin remodeling complex and the transcription factor can be examined using a GST-pulldown/immunoblotting analysis of protein interactions between recombinant forms of the proteins of interest as described in Example 6 and Kadam, et al., Genes & Dev. 14: 2441-51 (2000). See also 4 CURRENT PROTOCOLS IN MOLECULAR BIOLOGY 20.2. GST fusion proteins can be generated using methods well known in the art. In one example, the GST fusion protein is then purified on glutathione-agarose beads. The bead bound fusion protein is then used as “bait” to test for binding to a known protein which may be either purified or labeled by in vitro translation in the presence or absence of a test compound. Beads with bound protein are then subjected to centrifugation, the non-bead containing supernatant removed, and the beads washed several more times in the binding buffer (e.g., 50 mM potassium phosphate, pH 7.5, 150 mM KCl, 1 mM MgCl2, 10% (v/v) glycerol, 1% (v/v) Triton X-100, and protease inhibitor cocktail (available from commercial sources)). Then the supernatant and bead fractions are subjected to analysis on a SDS-polyacrylamide gel, and the result visualized using an autoradiogram or coomassie staining. Comparison of the bound versus unbound fractions in the bead and supernatant fractions, respectively, allows quantitation of the protein-protein interactions. In another embodiment, the protein-protein interactions between one or more subunits of the chromatin remodeling complex and a transcription factor can be examined in vivo using coprecipitation of proteins from whole cell extracts as described in Example 6 and Kadam, et al., Genes Dev. 14: 2441-51 (2000). See also 4 CURRENT PROTOCOLS IN MOLECULAR BIOLOGY 20.5. Native or epitope tagged proteins can be used for which specific antibodies are available. For example, GST, histidine, and FLAG tags can be used in these methods. Tagging of proteins for in vitro or in vivo assays can be accomplished using well known protocols in the art. See, e.g., 4 CURRENT PROTOCOLS IN MOLECULAR BIOLOGY 10.15, 20.2, & 20.3.

In another embodiment, the protein-protein interactions between one or more subunits of the chromatin remodeling complex and a transcription factor can be examined using Far Western analysis as described in Kadam, et al., Genes Dev. 14: 2441-51 (2000). See also 4 CURRENT PROTOCOLS IN MOLECULAR BIOLOGY 20.6. Simply stated, in a far western blot, one protein of interest is immobilized on a solid support membrane, and then probed with a non-antibody protein. Briefly, the protein sample to be analyzed is separated on a SDS-PAGE gel and then transferred to a solid support membrane (e.g., nitrocellulose or PVDF) by semidry electroblotting. After blotting, the membrane is blocked to reduce non-specific interactions. The membrane is then incubated with the protein probe, typically for 2 hours at room temperature, and then washed. The protein probe can be directly labeled using in vitro translation using radiolabel (e.g., ³⁵S methionine) or detected indirectly using labeled antibody. The membrane is then dried and examined using autoradiography or phosphor imager screen.

In one embodiment, the protein-protein interactions between the chromatin remodeling complex and the transcription factor can be examined in the presence of DNA. A specific DNA sequence can be provided in vitro and the ability of one or more proteins to bind to the specific DNA sequence can be examined by gel shift mobility assay as described in Kadam, et al., Genes Dev. 14: 2441-51 (2000). Briefly, the gel shift assay is performed by incubating a purified protein, or a complex mixture of proteins (e.g., nuclear or cell extracts), with a ³²P end-labeled DNA fragment containing the putative protein binding site (e.g., regulatory region of the gene of interest). After incubation, the resultant products are separated on a nondenaturing polyacrylamide gel and analyzed by autoradiography. The specificity of the DNA-binding protein for the labeled DNA region is established by competition experiments using unlabeled DNA fragments containing a known binding site for the protein of interest, or other unrelated, unlabeled DNA sequences.

In another embodiment, the interaction of the chromatin remodeling complex, the transcription factor, and chromatin can be determined using chromatin immunoprecipitation analysis. See Example 6. See also 4 CURRENT PROTOCOLS IN MOLECULAR BIOLOGY 21.3. Cells containing the chromatin having the gene of interest can be exposed to any reagent or protocol to modulate the expression of the gene of interest. For example, a cell can be induced to proliferate, differentiate, or otherwise be subjected to a cell signaling cascade. Any cell can be used with this method. In one embodiment, the cell can be mammalian. In a specific embodiment, the cell can be a MEL cell. Any regulatory sequence of a gene of interest that is bound by a transcription factor can be used in this method to assess chromatin remodeling (i.e., promoter targeting) by the transcription factor and the chromatin remodeling complex. After a suitable contact time, interacting proteins can be bound using formaldehyde crosslinking. The formaldehyde crosslinking can be performed for any suitable length of time. In one embodiment, the time for formaldehyde crosslinking is 15 minutes. Any suitable concentration of formaldehyde can be used for crosslinking. In one embodiment, the final concentration of formaldehyde is 1%. Any suitable temperature for crosslinking can be used. In one embodiment, the range of temperature can be from 25° C. to 37° C., with a temperature of 37° C. typically used. Any transcription factor or chromatin remodeling complex subunit of interest can be examined for which there is a specific antibody. The antibody can be a monoclonal or a polyclonal antibody. Any suitable PCR primer pairs can be used for amplification of the isolated genomic sequences containing the gene of interest.

Any suitable method can be used to determine the amount of chromatin remodeling. In one embodiment, the amount of chromatin remodeling is determined using mononucleosome disruption assay. In another embodiment, the amount of chromatin remodeling is determined using the micrococcal nuclease digestion as described in 4 CURRENT PROTOCOLS IN MOLECULAR BIOLOGY 21.1. In another embodiment, the amount of chromatin remodeling can be determined by examining the generation of DNase I hypersensitivity sites within a specific DNA sequence See Example 6. See also 4 CURRENT PROTOCOLS IN MOLECULAR BIOLOGY 21.4. For such assays, the DNA of interest is end-labeled, typically by a radiolabel, prior to chromatin assembly. Following incubation with the chromatin remodeling complex, transcription factors, the chromatin-assembled DNA is subjected to DNase I enzymatic digestion. Controls include, but are not limited to the complex alone and transcription factor alone. DNase I associates with the minor groove of the double helix and cuts DNA endonucleically, leaving a single stranded nick. Cleavage products are visualized by separation on DNA sequencing gels, followed by autoradiography of the gel. For intranucleosomal DNA, DNase I cleavage results in nicks that produce a ladder of cleavage products on a sequencing gel in which the bands are spaced by 10-11 basepairs. If the minor groove is protected by histones (i.e., not remodeled), the minor groove is not cut and a distinct pattern where one or more bands are absent on a sequencing gel. Remodeled chromatin assembled DNA results in regions of DNase I hypersensitivity (i.e., greater cleavage) because the protective molecules are removed or more mobile, allowing greater the cleaving enzyme greater access to the DNA. The chromatin complexes can also be analyzed using hydroxyl radical cleavage using methods known in the art. For in vitro analysis of remodeling, chromatin assembled DNA can be incubated with transcription factors using any convenient protocol. The chromatin assembled DNA can be contacted in any order or simultaneously with the transcription factor and the chromatin remodeling complex in any volume. In one embodiment, the volume ranges from 10 μl to 500 μl, with a volume of 20 to 40 μl typically used. Such contact can be carried out at any temperature. In one embodiment, the digestion is carried out at ambient temperature. In another embodiment, the digestion is carried out at 37° C. Contact between the chromatin assembled DNA, the chromatin remodeling complex, and the transcription factor can be carried out at any concentration of Mg²⁺ that is suitable for the enzymatic activity of the DNAase I. Any period of time can be used for contacting the chromatin assembled DNA, the chromatin remodeling complex, and the transcription factor. In one embodiment, the contact time is one to two minutes. Digestion reaction can be stopped by any convenient method. Purified DNA can be analyzed by primer extension as described below and in the Example.

Any suitable method can be used to determine the modulation of gene expression by examining the amount of transcription for the gene of interest. In one embodiment, the amount of transcription for a gene of interest is determined in vitro. See, e.g., Kadam, et al., Genes Dev. 14: 2441-51 (2000). Chromatin assembled DNA containing the gene of interest can be incubated with one or more transcription factors and a chromatin remodeling complex using any convenient protocol. Chromatin assembly complexes can be provided by Drosophila embryonic extracts, generated recombinantly, or some combination thereof. For example, supplementation of the Drosophila embryonic extract with core histones and ATP permit in vitro chromatin remodeling/transcription in the presence of cloned DNA and purified chromatin proteins. The incubation of the various components can be for any suitable time. In one embodiment, the proteins and chromatin are incubated for 30 minutes. The incubation can be at any suitable temperature. In one embodiment, the temperature can be 27° C. Transcription product can be detected and quantitated by any suitable protocol. In one embodiment, the purified mRNA transcripts were analyzed by primer extension analysis. Typically, primer extension employs a oligonucleotide that is complementary to a portion of the RNA of interest. The primer is end-labeled, hybridized to the RNA, and extended by reverse transcriptase using unlabeled deoxynucleotides to form a single-stranded DNA complementary to the template RNA. The resultant DNA is analyzed on a gel. In one embodiment, the gel is sequencing gel. The yield of the primer extension product reflects the abundance of the RNA. An exemplary protocol for primer extension can be found at 1 CURRENT PROTOCOLS IN MOLECULAR BIOLOGY 4.8. Any method known in the art can be used to quantitate the abundance of complementary DNA template using various primer labels.

Methods to Identify Compounds that Modulate Gene Expression by Chromatin Remodeling

Also provided herein are compounds that modulate gene expression by modulating chromatin remodeling as identified by the methods provided herein. The modulation of gene expression can be determined by examining the protein-protein interactions between chromatin remodeling complexes and a transcription factor, chromatin remodeling, and transcription of a gene of interest in the presence or the absence of a test compound. Therefore, the test compound is a modulator of gene expression for the gene of interest. If the test compound alters (i.e., increases or decreases) the protein-protein interactions between chromatin remodeling complexes and a transcription factor, chromatin remodeling, and transcription of a gene relative to the protein-protein interactions between chromatin remodeling complexes and a transcription factor, chromatin remodeling, and transcription of a gene in its absence, the test compound is one that modulates gene expression.

For example, an assay detecting an interaction of between mammalian SWI/SNF subunits and zinc motifs can be designed. First, it is initially determined which SWI/SNF subunits interact with a specific zinc finger domain-containing protein by co-immunoprecipitation analyses. One example is the EKLF-BRG-1 complex formation in the Example 1.

Second, a modified binding assay is devised for high-throughput drug screening by attaching a fluorescent “tag” to one protein, for example recombinant BRG-1, and adding this labeled protein to multi-well plates that have been coated with a specific concentration of the full length protein or its zinc finger motif. A “control” (the protein or zinc finger motif containing mutations that abolish SWI/SNF subunit binding as determined in the first step above) is also used to monitor any non-specific binding that may occur in these reactions.

Third, after a brief period of incubation for protein-protein interaction to occur, the stability of these complexes is challenged by screening a library of small molecule compounds for their ability to alter the affinity of zinc finger-SWI/SNF subunit binding.

Fourth, after a period of incubation with these compounds, the multi-well plates is extensively washed with appropriate buffer solutions and the fluorescence signal quantitated to give a precise measure of the increase or decrease in protein complex concentration in the presence of specific compounds. For example, the optimal amount of multi-well plate washing is determined by the amount needed to obtain a low fluorescent background with the control “mutant zinc finger” that should not bind SWI/SNF while preserving a high fluorescent signal with another control containing a “wild type zinc finger” that should bind SWI/SNF with high affinity.

Compounds that significantly increase or decrease transcription factor-SWI/SNF interactions are further examined in in vitro chromatin remodeling and transcription assays and then tested in cultured cells to verify their efficacy before animal or human testing. It is also possible that a specific combination of compounds will be most efficacious. The ultimate goal is not only for efficacy, but for specificity, such that the compound or compound cocktail enhances or interferes with its target transcription factors-SWI/SNF subunit interaction without enhancing or interfering with the activity of other transcription factors.

A non-comprehensive list of possible SWI/SNF subunits and transcription factors include the following: SWI/SNF subunits including, but not limited to BRG-1, hBRM, BAF 155, BAF 170, INi1, BAF 60, BAF 47, BAF 57; zinc finger proteins including, but not limited to GATA-1 (erythroid), Sp1 (ubiquitous), EKLF erythroid), FKLF (fetal), BKLF (basic), GKLF (gut), LKLF (lung), nuclear hormone receptors such as, androgen, estrogen, thyroid, progesterone, glucocorticoid receptors, Wilm's tumor suppressor protein (WT1), BRCA1, BRCA2, KRAB repressor domain-containing zinc-finger proteins, and BTB/POZ domain-containing zinc-finger proteins such as, PLZF (promyelocytic leukemia zinc finger).

This assay can also be used to screen for drugs that modulate the interaction between SWI/SNF subunits and transcription factor fragments and/or fusion proteins containing transcription factor fragments. These fragments and proteins can potentially function as gene-specific transcriptional regulators when introduced into cells.

A test compound is identified as a modulator of gene expression when it is capable of specifically increasing or decreasing the protein-protein interactions between chromatin remodeling complexes or portions thereof and a transcription factor or fragments thereof. The increase or decrease in the interaction in the presence of the compound is relative to appropriate controls which include, but are not limited to using protein-protein complex where SWI/SNF is not participating and the SWI/SNF complex plus an interacting protein that is unrelated to the transcription factor of interest. Additionally, interactions may be determined using protein-protein interactions alone or protein-protein-nucleosome interactions.

A test compound is identified as a modulator of gene expression when it is capable of specifically increasing or decreasing the amount of protein-protein interactions in the assays described herein or using assays well known in the art. The increase or decrease in the amount of protein-protein interactions is determined by a relative comparison to the amount of protein-protein interactions in the absence of the test compound. A compound is a modulator of gene expression if it increases or decreases the amount of protein-protein interactions by at least 5%, often by 20-50%. A compound may increase or decrease the amount of protein interactions by as much as 100%. Controls used to determine the amount of protein interactions in the absence of the test compound include, but are not limited to protein-proteins that do not include SWI/SNF complex or subunits thereof and SWI/SNF complex protein interactions that are unrelated to the transcription factor or fragment thereof.

A test compound is also identified as a modulator of gene expression when it is capable of specifically increasing or decreasing the amount of chromatin remodeling in the assays described herein or using assays well known in the art. The increase or decrease in the amount of chromatin remodeling of the DNA containing the gene of interest is determined by a relative comparison to the amount of chromatin remodeling of the DNA containing the gene of interest in the absence of the test compound. A compound is a modulator of gene expression if it increases or decreases the amount of chromatin remodeling by at least 5%, often by 20-50%. A compound may increase or decrease the amount of chromatin remodeling by as much as 100%. Controls used to determine the amount of chromatin remodeling in the absence of the test compound include, but are not limited to remodeling unrelated to SWI/SNF complex-mediated remodeling (i.e. mediated by another complex) and SWI/SNF remodeling that is unrelated to the transcription factor of interest.

A test compound is identified as a modulator of gene expression when it is capable of specifically increasing or decreasing the amount of transcription. Transcriptional activity can be quantitated using detectably labeled primers. A compound is a modulator of gene expression if it increases or decreases the amount of transcription by at least 5%, often by 20-50%. A compound may increase or decrease the amount of transcription by as much as 100%. Controls used to determine the amount of transcription in the absence of the test compound include, but are not limited to transcription unrelated to SWI/SNF complex-mediated transcription and SWI/SNF-induced transcription that is unrelated to the transcription factor of interest.

In one embodiment, the test compound specifically inhibits the binding of BRG-1 ATPase with a transcription factor comprising a zinc finger by binding or otherwise interacting with the N-terminal protein interaction domains not present in BRM ATPase. The N-terminal of BRG-1 ATPase is defined as amino acids 1-282 of the mature protein.

The amount of test compound that is present in the contact mixture may vary, particularly depending on the nature of the test compound. In one embodiment, where the agent is a small organic molecule, the amount of molecule present in the reaction mixture can range from about 1 femtomolar to 10 millimolar. In another embodiment, where the agent is an antibody or binding fragment thereof, the amount of the test molecule can range from about 1 femtomolar to 10 millimolar. The amount of any particular agent to include in a given contact volume can be readily determined empirically using methods known to those of skill in the art.

The test compound, chromatin assembled DNA, chromatin remodeling complex (or subunits), and transcription factor (or transcription factor fragment) as provided herein are contacted using any convenient protocol. In one embodiment, the test compound is contacted with a cell containing the chromatin assembled DNA, chromatin remodeling complex (or subunits), and transcription factor (or transcription factor fragment). The chromatin assembled DNA, chromatin-remodeling complex transcription factor, or any combination thereof can be endogenously- or exogenously-derived relative to the cell. The cell can be any cell containing a gene of interest. The cells and test compound can be contacted for any period of time where toxicity does not result. For example, the time of contact ranges from 1 minute to one hour, with a preferred time of 30 minutes. The cells and test compound can be contacted in medium at any pH. The cells and the test compound can be contacted at various temperatures. In one embodiment, the temperature for contact often ranges from 25° C. to 38° C., with a temperature of 37° C. typically utilized. When desirable, the cells and the test compound may be agitated to ensure adequate mixing and contact with the test compound. In another embodiment, the test compound, chromatin assembled DNA, chromatin remodeling complex (or subunits), and transcription factor (or transcription factor fragment) as provided herein are contacted in vitro (i.e., in the absence of intact cells). They can be placed into a container that can hold a volume of a fluid medium, e.g., a well of a 96-well plate or 384 well plate, or an analogous structure. They can be contacted in any volume will permit accurate detection of the measured events (i.e., protein-protein interaction, chromatin remodeling, transcription). In one embodiment, the reaction volume ranges from about 20 to 200 microliters. The cells and test compound can be contacted for any period of time. For example, the time of contact ranges from 1 minute to one hour, with a preferred time of 30 minutes. The cells and test compound can be contacted in medium at any pH permissive for the measured event. The cells and the test compound can be contacted at various temperatures. In one embodiment, the temperature for contact often ranges from 25° C. to 38° C., with a temperature of 37° C. typically utilized. When desirable, the acellular components can be agitated to ensure adequate mixing and contact with the test compound.

Test Compounds

A variety of different test inhibitory molecules may be identified using the method as provided herein. Compounds that are useful in the present invention include those that modulate (i.e., increase or decrease) gene expression by altering modulating chromatin remodeling. For example, this modulating activity of the compound can be a result of the small molecule interacting with the subunit of the chromatin remodeling complex, such as a SWI/SNF subunit, or with the domain within the transcription factor, such as a zinc-finger binding domain.

Test molecules can encompass numerous chemical classes. In certain embodiments, they are organic molecules, preferably small organic compounds having a molecular weight of more than 50 and less than about 2,500 daltons. Test molecules can comprise functional groups necessary for structural interaction with proteins, particularly hydrogen bonding, and may include at least an amine, carbonyl, hydroxyl or carboxyl group, preferably at least two of the functional chemical groups. The test molecules can comprise cyclical carbon or heterocyclic structures and/or aromatic or polyaromatic structures substituted with one or more of the above functional groups. Test molecules are also include biomolecules like peptides, polypeptides, peptidomimetics, saccharides, fatty acids, steroids, purines, pyrimidines, derivatives, structural analogs or combinations thereof. Test molecules of interest also can include peptide and protein agents, such as antibodies or binding fragments or mimetics thereof, e.g., Fv, F(ab′)₂ and Fab. Known pharmacological agents may be subjected to directed or random chemical modifications, such as acylation, alkylation, esterification, amidification, etc. to produce structural analogs.

The compounds useful in the present invention are found among biomolecules including, but not limited to peptides, saccharides, fatty acids, steroids, purines, pyrimidines, derivatives, structural analogs or combinations thereof. Alternatively, the compound could be an antibody. Appropriate compounds further include chemical compounds (e.g., small molecules having a molecular weight of more than 50 and less than 5,000 Daltons, such as hormones). Candidate organic compounds comprise functional groups necessary for structural interaction with proteins, particularly hydrogen bonding, and typically include at least an amine, carbonyl, hydroxyl or carboxyl group, preferably at least two of the functional chemical groups. The candidate organic compounds often comprise cyclical carbon or heterocyclic structures and/or aromatic or polyaromatic structures substituted with one or more of the above functional groups. Known pharmacological compounds are candidates which may further be subjected to directed or random chemical modifications, such as acylation, alkylation, esterification, amidification, etc., to produce structural analogs.

A variety of other compounds may be included in the method of the present invention. These include agents like salts, neutral proteins, e.g., albumin, detergents, etc. that are used to facilitate optimal protein-protein binding or interactions and/or reduce nonspecific or background binding or interactions. For example, reagents that improve the efficiency of the assay, such as protease inhibitors, nuclease inhibitors, antimicrobial agents, may be used. The mixture of components are added in any order that provides for the requisite modulation. Moreover, such compounds additionally can be modified so as to facilitate their identification or purification. Such modifications are well known to the skilled artisan (e.g., biotin and streptavidin conjugated compounds).

Test compounds include antibodies, peptides and chemical compounds including, but not limited to TSA, Na Butyrate, 5-aza-cytidine inhibits DNA methyltransferases.

Also featured is structural information descriptive of a chromatin remodeling modulator identified by the processes described herein. In certain embodiments, information descriptive of a chromatin remodeling modulator structure (e.g., chemical formula or sequence information) sometimes is stored and/or renditioned as an image or as three-dimensional coordinates. The information sometimes is stored and/or renditioned in computer readable form and sometimes is stored and organized in a database. In certain embodiments, the information may be transferred from one location to another using a physical medium (e.g., paper) or a computer readable medium (e.g., optical and/or magnetic storage or transmission medium, floppy disk, hard disk, random access memory, computer processing unit, facsimile signal, satellite signal, transmission over an internet or transmission over the world-wide web).

Formulations of Compounds and Uses Thereof

Any suitable formulation of compounds can be used in the methods provided herein. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts are obtained using standard procedures well known in the art, for example by reacting a sufficiently basic compound such as an amine with a suitable acid affording a physiologically acceptable anion. Alkali metal (e.g., sodium, potassium or lithium) or alkaline earth metal (e.g., calcium) salts of carboxylic acids also are made.

The compounds may be formulated as pharmaceutical compositions and administered to a mammalian host in need of such treatment. In one embodiment, the mammalian host is human. Any suitable route of administration may be used, including but not limited to oral, parenteral, intravenous, intramuscular, topical and subcutaneous routes.

In one embodiment, the compounds provided herein may be administered systemically, e.g., orally, in combination with a pharmaceutically acceptable vehicle such as an inert diluent or an assimilable edible carrier. They may be enclosed in hard or soft shell gelatin capsules, compressed into tablets, or incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid. Examples of useful dermatological compositions that can be used to deliver the compounds of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949. Generally, the concentration of the compound(s) of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day. The compound is conveniently administered in unit dosage form; for example, containing 5 to 1000 mg, conveniently 10 to 750 mg, most conveniently, 50 to 500 mg of active ingredient per unit dosage form. Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s). The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Methods to Modulate Gene Expression by Chromatin Remodeling

Provided herein is a method for modulating gene expression, which comprises contacting a chromatin assembled DNA containing a gene of interest with (a) a transcription factor containing a tryptophan cluster or a leucine zipper, or a fragment of the transcription factor, and (b) a SWI/SNF chromatin remodeling complex containing BRG-1 ATPase, or a portion of the remodeling complex. In one embodiment, the transcription factor contains a leucine zipper, or fragment thereof. In a specific embodiment, the transcription factor is c-fos, c-jun, or C/EBPα, or a fragment thereof. In another specific embodiment, the transcription factor is phosphorylated CREB, or a fragment thereof. In one embodiment, the gene of interest is a cAMP-responsive gene. In another embodiment, the transcription factor contains a tryptophan cluster, or a fragment thereof. In a specific embodiment, the transcription factor is IRF-1, or a fragment thereof.

In one embodiment, the transcription factor fragment contains one or more regions selected from the group consisting of a leucine zipper, a tryptophan cluster, a DNA binding domain, an activation domain, a regulatory domain, a protein interaction domain, or a fragment of the foregoing.

Further provided herein is a method for modulating gene expression, which comprises contacting a chromatin assembled DNA containing a gene of interest with (a) a SWI/SNF chromatin remodeling complex; (b) a first transcription factor or fragment thereof comprising an activation domain, wherein the first transcription factor or fragment thereof does not interact with the SWI/SNF chromatin remodeling complex comprising BRG-1 ATPase to initiate transcription, and (c) a DNA-binding domain or fragment thereof of a second transcription factor, wherein the DNA-binding domain or fragment thereof of the second transcription factor interacts with the remodeling complex thereby allowing access of the first transcription factor or fragment thereof to the gene of interest, whereby gene expression is modulated. In one embodiment, the SWI/SNF chromatin remodeling complex comprises BRG-1 ATPase. In a specific embodiment, the DNA-binding domain of the second transcription factor contains a zinc finger, or a fragment thereof. In another embodiment, the SWI/SNF chromatin remodeling complex comprises BRM ATPase.

Also provided herein is a method for modulating gene expression, which comprises contacting a chromatin assembled DNA containing a gene of interest with (a) a transcription factor containing an ankyrin repeat, or a fragment of the transcription factor; and (b) a SWI/SNF chromatin remodeling complex containing BRM ATPase, or a portion of the remodeling complex. In one embodiment, the transcription factor is ICD22 or CBF-1, or a fragment thereof. In one embodiment, the gene of interest is a Notch receptor regulated gene.

Further provided herein are high-throughput screening assays that identify small molecule compounds that enhance or block the association between chromatin remodeling complexes and the specific transcription factors with which they interact, such as the BRG-1 subunits of the SWI/SNF complex and various transcription factors. In this way, very specific drugs are developed that modulate the activation or repression of selective genes that are differentially regulated by SWI/SNF complexes containing distinct ATPases (e.g., BRG-1 ATPase and BRM ATPase). Specific drugs permitting targeted and highly selective modulation of gene expression also provide methods for treating genetic diseases where less specific drugs result in prohibitive toxicity or unacceptable side effects.

Further provided herein are high-throughput screening assays that identify small molecule compounds that enhance or block the association between chromatin remodeling complexes and the specific transcription factors with which they interact, such as the BRG-1 subunits of the SWI/SNF complex and various transcription factors. In this way, very specific drugs are developed that modulate the activation or repression of selective genes that are differentially regulated by SWI/SNF complexes containing distinct ATPases (e.g., BRG-1 ATPase and BRM ATPase). Specific drugs permitting targeted and highly selective modulation of gene expression also provide methods for treating genetic diseases where less specific drugs result in prohibitive toxicity or unacceptable side effects.

The following examples are intended to illustrate but not limit the invention.

### EXAMPLE 1

### Transcriptional Specificity of Human SWI/SNF BRG-1 and BRM Chromatin Remodeling Complexes

These studies investigated the basis by which BRG-1- and BRM-containing human SWI/SNF complexes regulate distinct genes by identifying members of protein families that can discriminate between the two ATPases and target each one to selected promoters. In this study, BRG-1, but not BRM, functions specifically with zinc finger proteins (ZFP). ZFPs represent the most common class of eukaryotic regulatory factors whose members control a wide range of cellular processes including differentiation, proliferation, metabolism, and apoptosis. Thus far, SWI/SNF is the only chromatin remodeling complex shown to mediate the activity of this important family of diverse proteins. Members of other protein families containing leucine zippers, rel domains, HMG boxes, tryptophan clusters, arm repeats, and ankyrin repeats either fail to interact with SWI/SNF or display high specificity for either BRG-1 or BRM complexes but not both. Interestingly, two critical components of the Notch signaling pathway associate exclusively with BRM SWI/SNF and recruit this complex to natural target promoters in vivo. Therefore, BRM ATPase may be specifically dedicated to modulating the chromatin structure of genes regulated by this particular pathway. An examination of the recruitment of SWI/SNF to a variety of genes during cellular proliferation, differentiation, or signal transduction reveals an exclusivity of BRG-1 or BRM association that occurs before transcriptional reprogramming. The final switch to activation or repression is mechanistically diverse among tissue-specific, signal-activated, cell cycle-regulated, and housekeeping genes, and can be correlated with changes in histone modification, activator binding, and relative ratios of CBP/HDAC.

Zinc Finger DNA binding Domains Generate Chromatin Accessibility to Proteins that do not Function with SWI/SNF

A zinc finger DNA binding domain (ZF DBD) alone is sufficient to target hSWI/SNF to specific nucleosomal sites for stable remodeling. However, transcription at this site also requires an activation domain fused to the ZF DBD (Kadam, S., et al., Genes Dev. 14:2441-2451 (2000)). Based upon this observation, this study examined whether ZF DBD recruitment of SWI/SNF to a chromatin-assembled promoter could generate a remodeled structure that was accessible to transcription factors which cannot normally bind to their nucleosomal sites and do not function with SWI/SNF. To address this question, this study focused on the helix-loop-helix protein, TFE-3, which is a potent transcriptional activator of the HIV-1 promoter (Sheridan, P. L., et al., Genes Dev. 9:2090-2104 (1995)) but does not function with hSWI/SNF (Armstrong, J. A., et al., Cell 95:93-104 (1998).; Kadam, S., et al., Genes Dev. 14:2441-2451 (2000)). Plasmids containing the HIV-1 promoter were assembled into chromatin using Drosophila embryonic extracts (Bulger, M., et al., Methods Mol. Genet. 5:242-262 (1994)) and examined by in vitro transcription and nucleosome remodeling. In vitro transcription of chromatin-assembled HIV-1 promoters employed 1 μg of chromatin was incubated in the presence or absence of hSWI/SNF and either 50 pmol of TFE-3 or 125 pmol of the EKLF ZF DBD. All factors were added after chromatin assembly (post-assembly) except in controls. Reactions were split in half and analyzed separately by transcription and footprinting. α-globin plasmids were transcribed as internal controls. Transcription analysis quantitation was performed by primer extension analysis. TFE-3 functions only when pre-bound to HIV-1 promoter DNA prior to chromatin assembly, resulting in the appearance of a transcript. If added after assembly, it cannot access its binding site at −177 nor activate transcription in the presence or absence of purified hSWI/SNF, resulting in an absence of a detectable transcript. Addition of the ZF DBD from the erythroid factor EKLF to TFE-3 also fails to coactivate HIV-1 transcription through a −50 CACC element. However, if hSWI/SNF is included with TFE-3 and the ZF DBD, robust transcription occurs which is dependent upon TFE-3 since the ZF DBD lacks an activation motif, as evidenced by a detectable band. hSWI/SNF does not coactivate transcription with the ZF DBD alone nor with TFE-3 in the absence of the ZF DBD. To correlate these results with chromatin remodeling, DNase 1 footprinting was performed on the assembled HIV-1 templates. In each lane, the control a globin transcript was detected, confirming TFE-3 alone does not bind to chromatinized HIV-1 promoters (i.e., no DNase I hypersensitivity sites observed) and SWI/SNF does not facilitate this interaction. The ZF DBD also fails to interact with its recognition site at −50 unless catalyzed by SWI/SNF. However, consistent with the transcription results, TFE-3 interacts with the nucleosomal HIV-1 promoter very strongly in the presence of both SWI/SNF and the ZF DBD (i.e., DNase I hypersensitivity sites observed) whereas the ZF DBD alone does not enable TFE-3 to bind.

TFE-3 belongs to a class of proteins that does not interact with SWI/SNF and whose binding to chromatin is not directly facilitated by this remodeling complex in vitro. Therefore, no targeted chromatin restructuring of the HIV-1 promoter was catalyzed by SWI/SNF in the presence of TFE-3. However, a ZF DBD, which is insufficient for transcription, recruited SWI/SNF through a −50 CACC site to the nucleosome-repressed promoter. This generated an accessible structure extending to at least −177 which enables TFE-3 to interact and subsequently activate transcription. These results support the notion that among the multiple proteins that regulate eukaryotic promoters and enhancers, only one may be required to initially target a remodeling complex to render a genetic element accessible to interact with other factors in a temporal or conditional manner.

Human SWI/SNF is Targeted to Chromatin through Specific Zinc Fingers of the KLF and GATA Protein Families that Preferentially Bind DNA

In this study, purified recombinant forms of each ZF within the C₂H₂ and C4 DBDs were used to examine their individual roles in targeted SWI/SNF remodeling and transcriptional activation.

DNase hypersensitivity experiments demonstrated that C4 and C₂H₂ ZF DBDs each function with hSWI/SNF to direct chromatin remodeling. DNase I hypersensitivity of chromatin-assembled β-globin promoters were generated by SWI/SNF and ZF DBDs from KLF, and Sp1, and individual GATA-1 ZFs using assembled chromatin incubated with hSWI/SNF and 35 pmol of each protein as shown before digestion with 2 and 1 U DNase1. Interestingly, when individual C4 zinc fingers of GATA-1 were examined in this assay only the C-finger could target SWI/SNF. Protein interaction studies by GST-pulldown experiments indicated that SWI/SNF exclusively associated with the GATA C-finger whereas no binding was observed to the N-finger. GST-pulldown analysis of protein interactions was performed using 2 μg recombinant BRG-1 and 1 μg individual ZFs (F1, F2, F3) within the KLF ZF DBD. BRG-1 was detected by immunoblotting using BRG-1 antisera in the bound versus supernatant fractions. A similar examination of SWI/SNF interaction with each of the three individual C₂H₂ KLF ZFs revealed that fingers 1 and 3 but not 2 interacted with the recombinant BRG-1 subunit. The importance of SWI/SNF interaction with specific KLF fingers was examined functionally by in vitro transcription of chromatin-assembled β-globin genes. In vitro transcription of chromatin-assembled β-globin promoters was performed using assembled chromatin incubated with the following proteins: WT-HIS, —HIS2=two preparations of histidine-tagged EKLF (37 pmol); F123=histidine-tagged EKLF DBD; GST F123=GST-tagged EKLF DBD; F1, F2, F3=individual KLF zinc fingers (250 pmol). AdLuc transcripts were used as internal controls. In these experiments, ZF DBD or individual ZF were incubated with EKLF and SWI/SNF for 15 min on ice before addition to assembled chromatin. In this case, individual fingers were tested for their ability to act as dominant negative inhibitors of EKLF-dependent activation. Fingers 1 or 3 individually repress transcription by intact EKLF whereas finger 2 has no effect. A corresponding footprint analysis of these chromatin templates revealed that EKLF interaction with the β-globin promoter was abolished by addition of fingers 1 and 3 but not by finger 2. These results indicated that SWI/SNF was targeted to chromatin by ZFPs through association with specific ZFs within C4 and C₂H₂ DBD motifs that were the most critical for DNA binding.

Specificity of BRG-1 vs. BRM SWI/SNF Complexes for Zinc Finger DNA Binding Domains

This study explored further the observed selectivity of hSWI/SNF for ZF-containing proteins by examining which particular form of SWI/SNF these factors associate with. Our purified preparation of hSWI/SNF was composed of both BRG-1- and BRM-containing complexes since a common subunit, INI1, was tagged. SDS-PAGE silver stain analysis identified the epitope-tagged native hSWI/SNF complexes purified from HeLa INI11 cells while the immunoblot analysis of hSWI/SNF using antisera to BRG-1 and BRM subunits show the presence of both complexes in our preparations. In order to discriminate between the BRG-1 and BRM complexes present in hSWI/SNF, protein interaction experiments were performed using hSWI/SNF and GST-tagged ZF DBDs. GST-pulldown analysis of protein interactions was performed using 3 μg native hSWI/SNF and 1 μg recombinant ZF DBDs of EKLF, BKLF, Sp1, retinoic acid receptors (RAR and RXR), GATA-1, and individual GATA-1 C- and N-ZFs. Interactions with BRG-1 or BRM were detected by immunoblotting using appropriate antisera. The ZF DBDs of KLF factors, Sp 1, hormone receptors (RAR, RXR), and GATA-1 each bound specifically to BRG-1-containing SWI/SNF. In the case of GATA-1, SWI/SNF associated through the C-finger, consistent with the role of this finger in nucleosome remodeling. This analysis was repeated using recombinant BRG-1 and BRM subunits and showed that interactions with various ZF DBDs occurred specifically with the BRG-1 ATPase. GST-pulldown analysis of protein interactions was performed using 500 ng recombinant BRG-1 or BRM and 500 ng recombinant ZF DBDs, individual ZFs, ICD22 and CBF-1. 100% of bound proteins (beads), 50% of unbound proteins (sup), and 50% of input BRG-1 and BRM were analyzed on 10% SDS-PAGE gels and immunoblotted with antibodies against BRG-1 or BRM. Our in vitro interactions were confirmed by in vivo immunoprecipitation using mouse erythroleukemia cell extracts. As expected, EKLF interacted strongly with SWI/SNF complexes containing BRG-1 but not BRM. In vivo interaction of EKLF with SWI/SNF were examined using co-immunoprecipitation of EKLF with antisera to BRG-1, BRM, BAF155 and BAF170 subunits of SWI/SNF using 100 μg mouse erythroid (MEL) cell nuclear extract. Immune complexes were separated by 10% SDS-PAGE and immunoblotted with antibodies against EKLF. Recombinant EKLF (25 ng) was used as a positive control. Antisera to the core subunits BAF 155 and BAF 170 also co-precipitated EKLF, apparently from BRG-1 complexes only. Therefore, the ZF family of transcription factors interacted exclusively with BRG-1-containing SWI/SNF, and this occurred through specific ZFs and the BRG-1 ATPase subunit. No binding was observed between the ZF DBDs examined and native or recombinant forms of BRM-containing SWI/SNF.

Native and Recombinant BRG-1 but not BRM SWI/SNF Complexes Function with Zinc Finger Proteins to Activate Transcription Through Chromatin

To confirm the functional significance of the interaction specificity between BRG-1-containing SWI/SNF and ZFPs, the ability of immunopurified native BRG-1 and BRM SWI/SNF and recombinant BRG-1 and BRM complexes were compared as coactivators of transcription by EKLF. Native BRG-1 SWI/SNF immunodepleted of BRM complexes and recombinant BRG-1/BAF155 each activated β-globin transcription by EKLF. EKLF-dependent transcription from chromatin-assembled β-globin promoters was examined using native INI-1 tagged SWI/SNF (containing both BRG-1 and BRM), immunopurified native BRG-1 or BRM SWI/SNF, and recombinant BRG-1 or BRM complexes. 100 ng of chromatin were incubated with 3.7 pmol EKLF, 20 ng F-BRG-1, 100 ng F-BRM, 100 ng of F-BAF155, 58 ng INI-1 tagged SWI/SNF, 60 ng or 120 ng immunopurified BRG-1- or BRM-containing SWI/SNF. However, no EKLF-dependent transcription was observed with native BRM SWI/SNF complexes or with recombinant BRM/BAF155. A mononucleosome disruption assay was performed with recombinant BRM to confirm that this protein preparation was active. Nucleosome disruption analysis showed that the recombinant BRM ATPase possesses classical chromatin remodeling activity and was performed using 300 ng of SWI/SNF, 100 ng and 500 ng of hBRM were incubated with reconstituted 5S mononucleosomes followed by DNase I digestion. Native BRG-1 and BRM SWI/SNF were examined by Western blotting to evaluate the efficiency of separation these complexes after immunodepletion. Equal amounts (100 ng) of native INI-1 tagged SWI/SNF and immunopurified BRG-1 or BRM SWI/SNF complexes were analyzed by Western blotting using antisera to BRG-1, BRM, BAF 155 and INI-1. These complexes were analyzed for their catalytic activity by measuring ATP hydrolysis. ATPase activity of SWI/SNF complexes was determined using approximately 55 ng of native SWI/SNF, immunopurified BRG-1 and BRM SWI/SNF complexes incubated in the presence or absence of 150 nM nucleosomes and (γ-³²P) ATP. The phosphate present at time zero is due to the presence of (γ-³²P) phosphate in the (γ-³²P) ATP stock. The ratio of inorganic phosphate to ATP was quantitated for each time point using a Molecular Dynamics Phospholmager. BRM SWI/SNF had lower ATPase activity than BRG-1 complexes, and comparable catalytic activities of all SWI/SNF complexes were analyzed in our in vitro transcription experiments. A micrococcal nuclease digest was performed on nucleosomal β-globin plasmids to assess the quality of chromatin assembly. Taken together, these results demonstrated that EKLF functionally discriminated between native and recombinant BRG-1 and BRM SWI/SNF complexes through interaction specificity and that the BRM catalytic subunit could not replace BRG-1 in ZFP-dependent transcription.

BRG-1 Specificity for Zinc Finger Proteins Occurs Through N-Terminal Protein Interaction Domains Which are not Present in BRM

The ability of EKLF to activate transcription with only SWI/SNF complexes containing the BRG-1 ATPase is particularly striking since BRG-1 and BRM share significant sequence homology (approximately 75% identical). BRG-1-N spans the N-terminal residues 1-282 and shows high sequence divergence from the corresponding region of BRM. BRG-1-C1 contains a 99 base pair exon that is unique to BRG-1, the conserved E7 sequence, and a portion of the lysine-arginine (KR) region. BRG-1-C2 spans the KR region and a bromodomain both of which are conserved in BRM. To address whether any of these regions within BRG-1 were responsible for the observed functional specificity with zinc finger proteins, we expressed and purified recombinant proteins containing each of the three distinct BRG-1 domains and performed GST-pulldown experiments to determine whether EKLF interaction occurred through one of these sequences.

The analysis revealed that EKLF interacted specifically with the N-terminal region of BRG-1 (BRG-1-N) which shares little sequence homology with BRM. GST-pulldown analysis of protein interactions was performed using 350 ng of his-tagged EKLF and 500 ng of each BRG-1 subdomain. Interactions were detected by immunoblotting using EKLF antisera. Purified EKLF was used as a positive control. The importance of this interaction was confirmed functionally by in vitro transcription and chromatin remodeling analyses by testing each of the three BRG-1 domains as competitive inhibitors. The BRG-1-N protein was a potent inhibitor of SWI/SNF-dependent EKLF activation of chromatin-assembled β-globin genes, presumably by interfering with the ability of EKLF to associate with this region of BRG-1 within the native complex. In vitro transcription of chromatin-assembled β-globin promoters was determined using assembled chromatin templates were incubated with EKLF (37 pmol per 1 μg of chromatin in a 100 μl reaction volume), SWI/SNF, and 300 pmol of each BRG-1 subdomain. Reactions were divided in half and used for transcription or DNase hypersensitivity. AdLuc transcripts were used as internal controls. The BRG-1-N protein similarly interfered with EKLF-targeted chromatin remodeling by SWI/SNF. DNase I footprint of chromatin-assembled β-globin promoters was performed to analyze the effect of recombinant BRG-1 subdomains as dominant negative inhibitors of SWI/SNF-dependent EKLF binding. Each chromatin reaction was again divided into 2 tubes of 150 ng and digested with 1 and 2 U DNaseI. Two other subdomains of BRG-1 that did not interact with EKLF (BRG-1-C1, —C2) had no effect on either transcription or chromatin remodeling. These results demonstrated that the functional specificity between ZFPs and BRG-1-containing SWI/SNF complexes was due to protein-protein interactions occurring through individual ZF within ZF DBD motifs and the N-terminal sequences of BRG-1 which were nonhomologous with BRM.

BRG-1 and BRM SWI/SNF Complexes Possess Unique Interaction Specificities with Different Protein Domains

To further understand the basis of factor selectivity, representative classes of different transcription factors were screened for the ability to preferentially interact with BRG-1- or BRM-containing native hSWI/SNF complexes. Some factors were synthesized by in vitro translation (c-fos, c jun, p50, LEF-HMG, C/EBPα, IFR-1, ELK DBD), and others were expressed as GST-fusion proteins (ELKF DBD, TFE-3, ICAT, 13-catenin, ICD22, CBF-1, hSWI/SNF). In vitro binding studies were performed using 500 ng native hSWI/SNF and 200 ng of the following in vitro translated proteins: c-fos, c-jun, heterodimer of c-fos/c-jun, NF-κB p50 DBD subunit, LEF HMG DBD, C/EBPα, IRF-1, and EKLF DBD. Immunoprecipitation was carried out using antisera to BRG-1, BRM and INI1 subunits of the hSWI/SNF complex and analyzed by SDS-PAGE. 10% of the in vitro translated protein was loaded as an input. GST-pulldown analysis of interactions were performed using 1 μg hSWI/SNF and 500 ng of the following GST-fused proteins: EKLF DBD, TFE-3, ICAT, β-catenin, ICD22, and CBF-1. Interactions were detected by immunoblotting using antibodies against SWI/SNF BRG-1, BRM, and INI1 subunits. Purified hSWI/SNF was used as a positive control. GST-pulldown and immunoblotting experiments revealed that some proteins fail to interact with hSWI/SNF whereas others associate preferentially or exclusively with BRG-1- or BRM-containing complexes. Proteins interacting with equal affinity to both BRG-1 and BRM SWI/SNF were not observed in any case analyzed.

Among the factors that failed to interact with hSWI/SNF were: TFE-3 (bHLH); components of the Wnt signaling pathway, ICAT and 13-catenin (ARM repeats); LEF-1 HMG DBD; and NF-κB p50 (rel DBD). Factors associating preferentially or exclusively with BRG-1 SWI/SNF complexes included: c-fos, c-jun, and C/EBPα (LEU zipper), IRF-1 (TRYP cluster), and, the EKLF ZF DBD. Interestingly, two proteins of the ankyrin repeat family showed a strong interaction preference for BRM rather than BRG-1 hSWI/SNF. In vivo interaction of CBF-1 with BRM-containing SWI/SNF were examined using co-immunoprecipitation of hSWI/SNF with antibodies to CBF-1 using whole cell extracts from Notch2-expressing mouse myoblasts (C2C12) and control cells. Immune complexes were separated by SDS-PAGE and analyzed by Western blots with antisera against BRG-1, BRM, and INI1 subunits of SWI/SNF. Native hSWI/SNF was used as a positive control. To confirm the in vitro observations, co-immunoprecipitation experiments were carried out using protein extracts from control and Notch2-expressing mouse myoblast cells using antibodies to CBF-1. CBF-1 interacted specifically with BRM-containing SWI/SNF complexes in both Notch2-expressing and -nonexpressing cells. No association between CBF-1 and BRG-1 SWI/SNF was detected. Thus, out of eight distinct protein domains examined for interaction specificity between BRG-1 and BRM, four did not associate with SWI/SNF (bHLH, ARM repeats, HMG, rel), three bind to BRG-1 complexes (LEU zip, TRYP cluster, ZF), and one showed marked preference for BRM complexes (ANK repeats). (All proteins that interacted with either BRG-1 or BRM SWI/SNF also co-precipitated with INI1 since it was a core subunit of both remodeling complexes.) Taken together, these results demonstrated that BRG-1 and BRM-containing SWI/SNF have distinct interaction preferences for regulatory proteins which can be classified on the basis of protein domain identity. The ability of mammalian SWI/SNF to discriminate among proteins in this manner provides a possible mechanism by which BRG-1 and BRM complexes are targeted to specific genes that control distinct cellular functions or pathways.

Promoter Localization of BRG-1 and BRM SWI/SNF Complexes and Chromatin Modifiers During Cellular Proliferation, Differentiation, and Signaling

To address whether different genes were targeted by BRG-1- or BRM-containing SWI/SNF, protein occupancies of a variety of tissue-specific and cell cycle regulatory genes during erythroid proliferation and differentiation in mouse erythroleukemia cells were examined. The β-globin gene, which was activated after chemically-induced differentiation; the Cdk inhibitors p16, p21, and p27 which were also upregulated; cyclin A and the housekeeping gene, DHFR, which were downregulated after differentiation; and the T cell receptora (TCRA) gene, which was permanently inactive in erythroid cells were examined. In addition to analyzing these promoters for BRG-1 and BRM interaction, the occupancy of other chromatin enzymatic complexes, the histone acetyltransferase CBP and the histone deacetylase HDAC1 were analyzed. The histone modification status within the proximal promoter of each gene was also determined.

Chromatin immunoprecipitation (ChIPs) analyses were conducted using antibodies to a variety of proteins including the transcriptional activators EKLF, in the case of the β-globin promoter, or Sp1, as a ubiquitous factor found on many promoters. Chromatin immunoprecipitation (ChIPs) analyses of SWI/SNF remodeling complexes (BRG-1, BRM, core subunit INI1), transcriptional activators (EKLF/Sp1, CBF-1, Notch2), chromatin coactivators/corepressors (HDAC I, CBP), and histone modifications (ACH3, ACH4, dimethyl lys4H3, core H3) were performed using a variety of promoters (left side) which are up- or downregulated or permanently inactivated (right side, indicated by arrows or by “X”) in the cell types examined. Briefly, formaldehyde crosslinked DNA was immunoprecipitated, using appropriate antibodies and the recovered DNA was employed as a template in PCR amplification using primers directed against different promoters. Input DNA (5%) and minus antibody controls were also included. The nucleosomes assembled on these promoters were acetylated on lysines 4 and 9 of histones H3 and H4 and dimethylated on H3 lysine 4. These modifications were characteristic of active genes. The major change occurring on these promoters upon upregulation after differentiation was the loss of HDAC1 and a decrease in bound BRG-1, presumably because the chromatin was already stably remodeled. In the p27 promoter, Sp1, BRG-1, and CBP was not bound in proliferating cells when expression was downregulated but was recruited after differentiation. This was correlated with a dramatic increase in H4 acetylation and H3 lys 4 dimethylation. DHFR was an example of a promoter that was apparently not regulated by SWI/SNF since neither BRG-1, BRM, nor INI1 were bound. Instead, when DHFR was upregulated in proliferating cells, Sp1 and CBP were recruited, H3 and H4 were acetylated and H3 was methylated. Upon differentiation, DHFR downregulation was associated with the loss of Sp1 and CBP, a decrease in histone acetylation and lys 4 dimethylation, and the appearance of HDAC 1. The cyclin A promoter was the only one in the erythroid cell survey that interacted with BRM. Interestingly, there was a switch in BRG-1 and BRM occupancy of this promoter since BRG-1 was predominantly bound during proliferation but only BRM remains after differentiation. The TCRαgene was permanently inactive in erythroid cells and was not associated with SWI/SNF subunits, CBP, acetylated or methylated histones, or even the corepressor HDACI.

The majority of promoters in our ChIPs analyses interacted with BRG-1 rather than BRM. This correlated with protein-protein interaction experiments which showed that most transcription factors examined, which may recruit SWI/SNF to different promoters, associate with BRG-1-containing complexes. BRG-1 interacted with an erythroid-specific gene, β-globin, and cell cycle regulatory genes, p16, p21, and p27 whereas both BRG-1 and BRM bind to cyclin A.

BRM preferentially associated with proteins that regulate the Notch signaling pathway, CBF-1 and ICD22. Upon signaling, the transmembrane Notch receptor was proteolytically cleaved to release the ICD (Notch intracellular domain) which translocated to the nucleus and formed a complex with CBF-1. This complex then activated Notch target genes which regulate a variety of cell fate decisions. See, e.g., Artavanis-Tsakonas, S., et al., Science 284:770-776 (1999); Anderson, A. C., et al., Curr. Opin. Genet. Dev. 11:554-560 (2001).

The physiological relevance of these protein interactions was determined by examining whether BRM was indeed recruited by CBF-1 to natural Notch target genes. ChIP analyses of Hes1 (hairy enhancer of split) and Hes5 promoters during Notch signaling were performed in mouse myoblasts. Amplification products were in the range of 400 bp and contain the CBF-1 sequence elements. Input DNA (5%) and minus antibody controls were also included. As shown by ChIPs analysis, CBF-1 bound two Notch-regulated promoters, Hes1 and Hes5, in mouse C2C12 myoblasts which expressed the Notch2 ICD as well as in control myoblasts. Significantly, BRM, but not BRG-1, was predominantly associated with these promoters in Notch2 expressing and non-expressing cells. This suggested that CBF-1 recruited SWI/SNF BRM complexes to Hes1 and Hes5 promoters through preferential interaction with BRM rather than BRG-1 and that SWI/SNF-dependent chromatin remodeling occurs before Notch signaling. INI1 was also bound to each promoter because it was a core subunit of all SWI/SNF complexes. The Hes1 and Hes5 promoters appeared to be poised for transcription by CBF-1 binding, BRM recruitment, and histone acetylation. Upon signaling, Notch2 associated with Hes promoters and provided an activation domain for CBF-1, which then stimulated transcription. Interestingly, another pronounced change after signaling was a switch in the relative amounts of promoter-bound CBP and HDAC1 which correlates with the induction of Hes gene expression.

Taken together, these results provided insight into the specificity of promoter recruitment of BRG-1 or BRM SWI/SNF complexes during proliferation, differentiation, and cell signaling. SWI/SNF interaction normally preceded transcription suggesting that nucleosome remodeling poised target promoters for eventual activation. With the exception of cyclin A, either BRG-1 or BRM, but not both, preferentially associated with individual promoters. BRG-1 bound to a tissue-specific gene and several cell cycle regulators whereas BRM interacted with cyclin A in combination with BRG-1 and exclusively with two genes that were induced by Notch signaling. Consistent patterns of SWI/SNF occupancy, nucleosome modifications, and relative CBP/HDAC levels among some promoters and marked differences in others were observed. This reflected the distinct levels at which genes are regulated in response to changes in cellular function.

Discussion

These studies revealed that human SWI/SNF complexes containing either BRG-1 or BRM as the catalytic subunit interacted specifically with different classes of regulatory proteins, enabling these enzymes to be selectively targeted to distinct sets of promoters to facilitate chromatin remodeling and transcription. This provided a mechanistic basis for members of these two broad categories of SWI/SNF to regulate different programs of gene expression. Representatives of the zinc finger family of proteins (ZFPs) interacted exclusively with BRG-1-containing SWI/SNF and recruited these complexes to specific chromatin sites. The ability of SWI/SNF to coactivate ZFP was first shown with nuclear hormone receptors (Yoshinaga, S. K., et al., Science 58:1598-1604 (1992)). The ZFP family is the largest class of transcription factors and many members have diverse roles in critical cellular processes. The ZF domain is a ubiquitous structural element that exists in several major motifs. For example, the C₂H₂ motif is the most abundant eukaryotic DNA binding element which is estimated to be present in 600-700 proteins. This study determined that SWI/SNF interacted with ZFPs through the ZF DBD and the BRG-1 ATPase. The basis for the observed specificity between ZFPs and BRG-1 complexes was that interaction occurred within a domain of BRG-1 that was nonhomologous with BRM. The role of individual ZFs within two structural motifs, C₂H₂ and C4, in mediating BRG-1 SWI/SNF function was then examined. Using the erythroid factors EKLF and GATA-1 as representative proteins that contain C₂H₂ and C4 domains, respectively, these studies demonstrate that BRG-1 binds to individual ZFs which are the most critical for DNA binding. This may seem paradoxical, however, ZF DBDs have been shown to associate with both RNA and protein. The EKLF and GATA-1 DBDs interacted with a variety of cofactors, often through specific ZFs (Cantor, A. B., et al., Oncogene 21:3368-3376 (2002)). The significance of such critical protein-protein interactions, including that of SWI/SNF, occurring through domains that must also bind DNA has yet to be elucidated.

The involvement of mammalian SWI/SNF in cell proliferation and differentiation has been shown in several systems. SWI/SNF cooperates with C/EBPa to control genes that specify either myeloid or adipocyte lineages (Kowenz-Leutz, E., et al., Mol. Cell 4:735-743 (1999); Pedersen, T. A., et al., Genes Dev. 15:3208-3216 (2001)) and MyoD to regulate muscle cell determination (de la Serna, et al., Nat. Genet. 27:187-190 (2001)). The role of SWI/SNF in mediating Rb-dependent cell cycle progression is well-documented (Harbour, J. W., et al., Curr. Opin. Cell Biol. 12:685-689 (2000)). Additionally, recent studies demonstrate that SWI/SNF influences thymic development by CD4 silencing and CD8 activation through separate contributions of BRG-1 and BAF57 (Chi, T. H., et al., Nature 418:195-199 (2002)). The analysis described herein of SWI/SNF promoter localization in differentiating erythroid cells shows that these complexes bind to a variety of tissue-specific and cell cycle regulatory genes. SWI/SNF recruitment generally occurs during proliferation indicating that the promoters surveyed were already poised for changes in transcriptional activity which will be induced by differentiation. It is apparent that different strategies are used to regulate gene activity during this specific window of transition. Some promoters have dramatic changes in histone modifications that correlate with altered gene expression whereas others show a clear gain or loss of activators, coactivators, and corepressors or a combination of both processes. The fascinating diversity of transcriptional mechanisms and the temporal order of specific regulatory events have been demonstrated in several elegant studies (Cosma, M. P., Mol. Cell 10:227-236 (2002)). In this analysis, BRG-1 or BRM, but not both, exclusively interact with most SWI/SNF-responsive promoters. This correlated well with the protein-protein interaction experiments which reveal that among the factors examined the majority associate preferentially with BRG-1 or BRM or have no affinity for either form of SWI/SNF. In no case was equivalent binding to both BRG-1 and BRM observed. It is possible that the recombinant and in vitro-translated forms of these proteins fail to interact without other cofactors or modifications. However, the clear binding preferences observed for a number of factors strongly support the notion that gene-targeted SWI/SNF activity can be regulated by highly specific protein interactions which discriminate between the two ATPases.

The BRM ATPase is expressed at high levels in differentiating cells yet the functional role of this protein and the identity of the genes it regulates are poorly understood. In this regard, the observation that two components of the Notch signaling pathway, CBF-1 and ICD22, strongly associate with BRM but not BRG-1 is especially intriguing. This pathway controls cell fate commitment in a broad range of developmental processes. In this study, CBF-1 recruited BRM to two natural target genes, Hes1 and Hes5, in myoblasts before Notch induction. This indicated that these promoters are already in a remodeled configuration and accessible to bind the activator, Notch2, upon signaling. Interestingly, these studies show that components of the Wnt signaling pathway, β-catenin and ICAT, fail to interact with either BRG-1 or BRM-containing SWI/SNF. This suggested that different requirements may exist for specific remodeling complexes to mediate the effects of distinct types of extracellular signals. In support of this, recent reports show that BRG-1-containing SWI/SNF is involved in regulating genes induced by cytokine pathways through interferon-α or -γ (Liu, H., et al., Mol. Cell. Biol. 22:6471-6479 (2002); Pattenden, S. G., et al., EMBO J. 21:1978-1986 (2002)). In addition to Hes1 and Hes5, the only other reported BRM target genes are cyclin A which is repressed in an Rb-dependent manner during the cell cycle (Dahiya, A., et al., Mol. Cell 8:557-569 (2001)) and α₁-antitrypsin (α₁-AT) which is activated upon enterocyte differentiation (Soutoglou, E., et al., Science 295:1901-1904 (2002)). Thus, a common feature of these BRM target genes is that they promote cellular differentiation and may have evolved to respond to BRM because its levels are more highly regulated in the cell than BRG-1.

Experimental Procedures

Cell Lines and Plasmid Constructions

Mouse erythroleukemia (MEL) cells were grown in a MEM, 10% calf serum, 1× pen-strep-L-glutamine and induced using 2% DMSO for 5 days. C2C12 mouse myoblasts expressing Notch2 ICD, were maintained in DMEM containing 20% FBS in the presence of 400 μg G418. Control C2C12 cells were maintained only in DMEM plus FBS and split every two days. Cell confluency did not exceed 70%. 13-CAT, HIV-1-Luc, EKLF, GATA-1, NF-κB, TFE-3, and Sp1 plasmids were constructed as described in Kadam et al., Gene Dev. 14:2441-51 (2000). BRG-1-N, —C1 and —C2 were cloned into the EcoRI and BamHI sites of pGEX-KG expression vector by introducing amino acids 1-282, 1235-1408, 1371-1573 of BRG-1 downstream of GST.

Protein Purification

Histidine-tagged or GST-fusion wild type and mutant proteins were expressed in E. coli BL21(DE3) pLysS or BL21(DE3) cells (Kadam, S., et al., Genes Dev. 14:2441-2451 (2000)). Some proteins were in vitro-translated with the Invitrogen transcription and translation system using ³⁵S (Amersham) as per the manufacturer's instructions. Human flag-tagged SWI/SNF, F-BRG-1, F-hBRM, F-BAFI55, and F-BAF170 were purified as described (Phelan, M. L., et al., Mol. Cell 3:247-254 (1999)). HeLa FL-INI1-11 cells were grown by the National Cell Culture Center. BRG-1 and BRM SWI/SNF complexes were immunopurified from 5 μg native hSWI/SNF using antisera to either BRG-1 or BRM as described (Yie, J., et al., Proc. Natl. Acad. Sci. 96:13108-13113 (1999)). The specific complexes were separated using Sepharose Protein-A beads and purified forms of SWI/SNF in the supernatants were assayed for activity. To measure the ATPase activity of SWI/SNF complexes, approximately 55 ng of purified complexes were incubated with or without 150 nM of nucleosomes. Reactions were initiated with 10 μM ATP, 7 mM MgCl₂ and a trace quantity of (γ-³²P) ATP in a final volume of 10 μl. At specific times after initiation of the reaction, 2 μl aliquots were quenched in 5 μl of stop solution containing 3% SDS, 100 mM EDTA and 50 mM Tris (pH 7.5). Each time point was spotted onto PEI-Cellulose TLC plates (EM Science). Inorganic phosphate was separated from unreacted ATP by running the TLC plates in 0.5 M LiCl, 1 M formic acid. The ratio of inorganic phosphate to ATP at each time point was quantitated using a Molecular Dynamics Phospholmager.

Chromatin Assembly and In Vitro Transcription

Chromatin was reconstituted using Drosophila embryonic extracts as described (Bulger, M., et al., Methods Mol. Genet. 5:242-262 (1994)). Following assembly, the chromatin template (1 μg in 100 μl) was incubated with wild type or mutant proteins, as indicated in the figure legends, and SWI/SNF for 30 min at 27° C. The reactions were then split in half and used for either transcription or structural analyses as described (Kadam, S., et al., Genes Dev. 14:2441-2451 (2000)).

Chromatin Structural Analyses

DNase 1 hypersensitivity reactions were performed as described in Kadam et al., Genes Dev. 14:2441-2451 (2000). Micrococcal nuclease digestions: 15 U enzyme were added to 500 μg of chromatin and 150 μg aliquots were processed at timed intervals and analyzed on 1.5% agarose gels. DNase I footprinting: after incubation of chromatin templates with specific factors, samples were digested with 33 U/ml of DNase I (Boehringer Mannheim) for 80 s at 27° C. Purified DNA fragments were analyzed by primer extension. Mononucleosome disruption: sequence-positioned mononucleosomes were reconstituted on 5S DNA by salt dialysis and subjected to sucrose gradient sedimentation as described (Armstrong, J. A., et al., Cell 95:93-104 (1998)).

Protein-Protein Interactions

Pulldown assays with GST or Histidine-fusion proteins were carried out as described (Kadam, S., et al., Genes Dev. 14:2441-2451 (2000)). Immunoprecipitation of 0.2-1 μg in vitro translated proteins and 0.5-3 μg SWI/SNF was performed using 0.5 μg of primary antibody against BRG-1, hBRM or INI1 followed by binding to Protein A- and G-agarose beads.

Chromatin Immunoprecipitation Assays

Chromatin immunoprecipitation assays were performed essentially as described (Liu, et al., Cell 106, 309-318 (2001)). 5 μg of antibodies to the following proteins: H3, ACH3, ACH4, Dimethyl H3 lys 4, CBP, HDAC1, EKLF, Sp1, hBRM, BRG-1, and INI1 (purchased from Upstate and Santa Cruz) were used according to the manufacturer's recommendations with some minor modifications. Immunoprecipitated promoter fragments were detected by PCR amplification.

### EXAMPLE 2

### Mammalian SWI/SNF Interacts with Phosphorylated CREB

To determine the ability of SWI/SNF to interact with phosphorylated CREB, GST pull-down assays were performed with 3 μg human SWI/SNF and 1 μg GST-fused Phosphorylated and unphosphorylated CREB. The SWI/SNF BRG-1 subunit was then detected by Western blot analyses using the antisera at 1:1000 dilution. EKLF DBD and phosphorylated CREB bound SWI/SNF BRG-1 subunit while no binding was observed with unphosphorylated CREB. SWI/SNF BRM subunit did not bind EKLF DBD, phosphorylated CREB or unphosphorylated CREB.

Modifications may be made to the foregoing without departing from the basic aspects of the invention. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow.

Citation of the above publications or documents is not intended as an admission that any of the foregoing is pertinent prior art, nor does it constitute any admission as to the contents or date of these publications or documents. U.S. patents and other publications referenced herein are hereby incorporated by reference.

1. A method for identifying a compound that modulates gene expression, which comprises:
(a) contacting a chromatin assembled DNA containing a gene of interest with
(1) a SWI/SNF chromatin remodeling complex containing BRG-1 ATPase, or a portion of the remodeling complex,
(2) a transcription factor containing a tryptophan cluster or a leucine zipper, or a fragment of the transcription factor, and
(3) a test compound; and
(b) determining whether there is an increase or decrease in chromatin remodeling of the DNA containing the gene of interest in the presence and absence of the test compound, whereby a test compound that increases or decreases chromatin remodeling relative to the chromatin remodeling in the absence of the test compound is identified as a compound that modulates gene expression, or
determining whether there is an increase or decrease in transcription of the gene of interest in the presence and absence of the test compound, whereby a test compound that increases or decreases transcription relative to the transcription in the absence of the test compound is identified as a compound that modulates gene expression.

2. The method of claim 1, wherein the increase or decrease in chromatin remodeling is determined by analyzing one or more DNaseI hypersensitive sites in the DNA containing the gene of interest.

3. The method of claim 1, wherein the transcription factor contains a leucine zipper, or a fragment thereof.

4. The method of claim 3, wherein the transcription factor is c-fos, cjun, or C/EBPα, or a fragment thereof.

5. The method of claim 3, wherein the transcription factor is phosphorylated CREB, or a fragment thereof.

6. The method of claim 1, wherein the gene of interest is a cAMP-responsive gene.

7. The method of claim 1, wherein the transcription factor contains a tryptophan cluster, or a fragment thereof.

8. The method of claim 7, wherein the transcription factor is IRF-1, or a fragment thereof.

9. A method for identifying a compound that modulates gene expression, which comprises:
(a) contacting a chromatin assembled DNA containing a gene of interest with
(1) a SWI/SNF chromatin remodeling complex containing BRG-1 ATPase, or a portion of the remodeling complex,
(2) a transcription factor fragment consisting of a C4 motif or C₂H₂ motif from a zinc finger domain, and
(3) a test compound; and
(b) determining whether there is an increase or decrease in chromatin remodeling of the DNA containing the gene of interest in the presence and absence of the test compound, whereby a test compound that increases or decreases chromatin remodeling relative to the chromatin remodeling in the absence of the test compound is identified as a compound that modulates gene expression, or
determining whether there is an increase or decrease in transcription of the gene of interest in the presence and absence of the test compound, whereby a test compound that increases or decreases transcription relative to the transcription in the absence of the test compound is identified as a compound that modulates gene expression.

10. The method of claim 9, wherein the increase or decrease in chromatin remodeling is determined by analyzing one or more DNaseI hypersensitive sites in the DNA containing the gene of interest.

11. A method for identifying a compound that modulates gene expression, which comprises:
(a) contacting a chromatin assembled DNA containing a gene of interest with
(1) a SWI/SNF chromatin remodeling complex containing BRM ATPase, or a portion of the remodeling complex,
(2) a transcription factor or fragment thereof, and
(3) a test compound; and
(b) determining whether there is an increase or decrease in chromatin remodeling of the DNA containing the gene of interest in the presence and absence of the test compound, whereby a test compound that increases or decreases chromatin remodeling relative to the chromatin remodeling in the absence of the test compound is identified as a compound that modulates gene expression, or
determining whether there is an increase or decrease in transcription of the gene of interest in the presence and absence of the test compound, whereby a test compound that increases or decreases transcription relative to the transcription in the absence of the test compound is identified as a compound that modulates gene expression.

12. The method of claim 11, wherein the increase or decrease in chromatin remodeling is determined by analyzing one or more DNaseI hypersensitive sites in the DNA containing the gene of interest.

13. The method according to claim 11, wherein the transcription factor contains an ankyrin repeat.

14. The method according to claim 13, wherein the transcription factor is ICD22 or CBF-1, or a fragment thereof.

15. The method according to claim 11, wherein the gene of interest is a Notch receptor regulated gene.

16. A compound identified by the method of claim 1 that modulates gene expression.

17. A compound identified by the method of claim 9 that modulates gene expression.

18. A compound identified by the method of claim 11 that modulates gene expression.
